(19) United States
(12) Patent Application Publication
(10) Pub. No.: US 2023/0257373 A1
(43) Pub. Date: Aug. 17, 2023

(54) QUORUM SENSING INHIBITORS AND METHODS OF USE

(71) Applicant: The Trustees of Indiana University, Bloomington, IN (US)

(72) Inventors: Julia Catherine Van Kessel Bochman, Bloomington, IN (US); Laura Caroline Brown, Bloomington, IN (US)

(21) Appl. No.: 18/041,241

(22) PCT Filed: Aug. 13, 2021
(86) PCT No.: PCT/US2021/046038

§ 371 Date: Feb. 10, 2023

Related U.S. Application Data
(60) Provisional application No. 63/064,965, filed on Aug. 13, 2020.

(51) Int. Cl. C07D 417/04; C07D 405/14; C07D 409/12; C07D 409/06; C07D 405/06; C07D 233/84; C07D 333/34

(52) U.S. Cl. CPC C07D 417/04; C07D 405/14; C07D 409/12; C07D 409/06; C07D 405/06; C07D 233/84; C07D 333/34

(57) ABSTRACT

Inhibition of quorum sensing in Gram-negative bacteria, particularly strains of Vibrio, by certain compounds is disclosed. Compositions to treat bacterial infections and methods to treat such infections are also provided.

### CROSS REFERENCE TO RELATED APPLICATIONS

This application claims priority to U.S. provisional patent application Ser. No. 63/064,963 filed on Aug. 13, 2020. The disclosure of the prior application is considered part to of the disclosure of the application, and is incorporated in its entirety into this application.

### GOVERNMENT SUPPORT CLAUSE

This invention was made with government support under GM124698 and TR001108 awarded by National Institutes of Health. The government has certain rights in the invention.

### FIELD OF THE INVENTION

Aspects of the invention relate to molecules that influence quorum sensing in certain Gram-negative bacteria found in marine environments and method of using the same to control bacterial populations.

### BACKGROUND

Vibrio species are Gram-negative bacteria that occur naturally in marine environments. Upon infection by Vibrios, an infected aquatic animal may display lethargy, loss of appetite, and/or have necrotic sores. To date, a common method to prevent a Vibrio infection and to treat an aquatic animal infected by Vibrios is to provide antibiotics in the water. However, antibiotic resistance may arise through the overuse, or improper use, of antibiotics.

Because the capture of aquatic animals is insufficient to meet global fish demand, aquaculture production is expected to remain an important part of fishery industry. In aquaculture production, disease outbreaks are considered to be a key constraint in the fish farming sector, resulting in significant losses. Disease outbreaks cause economic damage and reduced productivity and antibiotic use in aquaculture is a sensitive social issue, so the production of high-quality safe aquatic products seems to be in doubt. A global need exists for a method to prevent and to treat diseases of cultured fish and other aquatic animals that does not rely on antibiotics

The present invention addresses this need by providing compounds and methods that target quorum sensing circuits and inhibit quorum sensing in Gram-negative bacteria, particularly of Vibrio species.

### SUMMARY

The quorum sensing signaling systems in Vibrio bacteria converge to control levels of the master transcription factors LuxR/HapR, a family of highly conserved proteins that regulate gene expression for bacterial behaviors. A compound library screen identified 2-thiophenesulfonamide compounds that specifically inhibit Vibrio campbellii LuxR but do not affect cell growth. We synthesized a panel of 50 thiophenesulfonamide compounds to examine the structure-activity relationship effects on Vibrio quorum sensing. The most potent molecule identified, PTSP (3-phenyl-1-(thiophen-2-ylsulfonyl)-1H-pyrazole), inhibits quorum sensing in multiple strains of Vibrio vulnificus, Vibrio parahaemolyticus, and V. campbellii at nanomolar concentrations. However, thiophenesulfonamide inhibition efficacy varies significantly among Vibrio species: PTSP is most inhibitory against V. vulnificus SmcR, but V. cholerae HapR is completely resistant to all thiophenesulfonamides tested. Reverse genetics experiments show that PTSP efficacy is dictated by amino acid sequence in the putative ligand binding pocket: F75Y and C170F SmcR substitutions are each sufficient to eliminate PTSP inhibition. Further, in silico modeling distinguished the most potent thiophenesulfonamides from less effective derivatives. Our results revealed the previously unknown differences in LuxR/HapR proteins that control quorum sensing in Vibrio species and underscore the potential for developing thiophenesulfonamides as specific quorum sensing-directed treatments for Vibrio infections.

In one aspect, the disclosure relates to a compound of Formula (I)

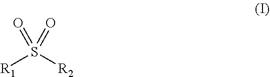

where R₁ and R₂ are independently selected from: an amino, an azide, a phenylamino, an unsubstituted or an optionally substituted aryl, an unsubstituted or an optionally substituted heterocyclic ring, or an acceptable salt thereof.

In another aspect, the disclosure relates to a compound selected from the group consisting of: (1H-pyrazol-1-yl)(thiophen-2-yl)methanone; furan-2-yl(1H-pyrazol-1-yl)methanone; (1H-pyrrol-1-yl)(thiophen-2-yl)methanone; furan-2-yl(1H-pyrrol-1-yl)methanone; 4-(4-phenylthiazol-2-yl)-2-(trifluoromethyl)morpholine; (4-(benzo[d][1,3]dioxol-5-ylmethyl)piperazin-1-yl)(5-((4-chlorophenoxy)methyl)furan-2-yl)methanone; (1H-imidazol-1-yl)(thiophen-2-yl)methanone; furan-2-yl(1H-imidazol-1-yl)methanone; 5-bromo-N′-((5-bromothiophen-2-yl)sulfonyl)thiophene-2-sulfonohydrazide; N′-(thiophen-2-ylsulfonyl)thiophene-2-sulfonohydrazide; and, 5-chloro-N′-((5-chlorothiophen-2-yl)sulfonyl)thiophene-2-sulfonohydrazide or an acceptable salt thereof.

In another aspect, the disclosure relates to a method to inhibit quorum sensing in Vibrio bacteria, the method comprising: contacting the bacteria or an environment containing the bacteria with a compound that inhibits LuxR activation and/or repression but does not affect growth of the Vibrio bacteria.

In another aspect, the disclosure relates to a method to treat a Vibrio infection in a subject in need thereof, the method comprising the step of administering to the subject a compound of Formula (I)

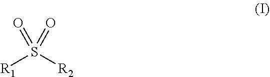

where R₁ and R₂ are independently selected from: an amino, an azide, a phenylamino, an unsubstituted or an optionally substituted aryl, an unsubstituted or an optionally substituted heterocyclic ring, or an acceptable salt.

In another aspect, the disclosure relates to a method to treat or to decrease a probability for developing a Vibrio species infection in an aquatic animal in need thereof, the method comprising: administering to the aquatic animal a therapeutically effective amount of a compound of Formula (I)

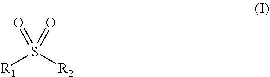

where R₁ and R₂ are independently selected from: an amino, an azide, a phenylamino, an unsubstituted or an optionally substituted aryl, an unsubstituted or an optionally substituted heterocyclic ring, or an acceptable salt.

Without limiting the scope of the invention, the following schemes, preparations, and examples are provided to further illustrate the invention. In addition, one of ordinary skill in the art appreciates that the compounds of Formula 1 may be prepared by using starting material with the corresponding stereochemical configuration which can be prepared by one of skill in the art.

The invention of the present disclosure can be described as embodiments in any of the following enumerated clauses. It will be understood that any of the embodiments described herein can be used in connection with any other embodiments described herein to the extent that the embodiments do not contradict one another,

In another aspect, the disclosure relates to a method to treat or to decrease a probability for developing a Vibrio species infection in an aquatic animal in need thereof, the method comprising: administering to the aquatic animal a therapeutically effective amount of a compound selected from the group consisting of: (1H-pyrazol-1-yl)(thiophen-2-yl)methanone; furan-2-yl(1H-pyrazol-1-yl)methanone; (1H-pyrrol-1-yl)(thiophen-2-yl)methanone; furan-2-yl(1H-pyrrol-1-yl)methanone; 4-(4-phenylthiazol-2-yl)-2-(trifluoromethyl)morpholine; (4-(benzo[d][1,3]dioxol-5-ylmethyl)piperazin-1-yl)(5-((4-chlorophenoxy)methyl)furan-2-yl)methanone; (1H-imidazol-1-yl)(thiophen-2-yl)methanone; 5-bromo-N′-((5-bromothiophen-2-yl)sulfonyl)thiophene-2-sulfonohydrazide; N′-(thiophen-2-ylsulfonyl)thiophene-2-sulfonohydrazide; and, 5-chloro-N′-((5-chlorothiophen-2-yl)sulfonyl)thiophene-2-sulfonohydrazide and, furan-2-yl(1H-imidazol-1-yl)methanone, or an acceptable salt thereof.

Clause 1. A compound of Formula (I)

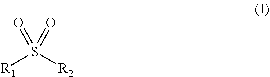

where R₁ and R₂ are independently selected from: an amino, an azide, a phenylamino, an unsubstituted or an optionally substituted aryl, an unsubstituted or an optionally substituted heterocyclic ring, or an acceptable salt thereof.

Clause 2. A compound according to clause 1, where R₁ is an optionally substituted aryl that is phenyl or a halogen substituted phenyl.

Clause 3. A compound according to clause 1, where R₁ is an optionally substituted heterocyclic ring that is an optionally substituted four to seven membered heterocyclic ring having at least one heteroatom.

Clause 4. A compound according to clause 3, where the optionally substituted heterocyclic ring is an alkyl substituted heterocyclic ring, an alkoxy substituted heterocyclic ring, a hydroxyl substituted heterocyclic ring or a halogen substituted heterocyclic ring.

Clause 5. A compound according to clause 3, where the four to seven membered optionally substituted heterocyclic ring is a five membered optionally substituted heterocylic ring having at least one heteroatom.

Clause 6. A compound according to clause 3, where the four to seven membered optionally substituted heterocyclic ring is a six membered optionally substituted heterocylic ring having at least one heteroatom.

Clause 7. A compound according to clause 1, where R₁ is an optionally substituted heterocyclic ring that is an optionally substituted thiophene, optionally substituted furan, optionally substituted pyrrolidine, optionally substituted pyrazole, optionally substituted imidazole, optionally substituted triazole, or optionally substituted piperidine.

Clause 8. A compound according to clause 1, where R₂ is an optionally substituted heterocylic ring that is an optionally substituted thiophene, a halogen substituted thiophene, an optionally substituted phenyl, phenyl, a halogen substituted phenyl, or a furan.

Clause 9. A compound according to clause 1, where the compound is not [1-(5-bromothiophene-2-sulfonyl)-1H-pyrazole.

Clause 10. A composition comprising a compound according to clause 1 and an acceptable carrier.

Clause 11. A composition according to clause 10, where the composition is formulated as a fish feed or fish feed additive.

Clause 12. A composition according to clause 10, where the composition is a solid composition.

Clause 13. A composition according to clause 10, where the composition is formulated as a fish feed additive and comprises one or more compounds selected from the group consisting of: maltodextrin and trehalose.

Clause 14. A composition according to clause 11, where the fish feed additive is freeze-dried.

Clause 15. A composition according to clause 10, where the composition is formulated as a fish feed and further comprises fish meal.

Clause 16. A compound according to clause 1, where the compound is selected from the group consisting of: 1-((5-bromothiophen-2-yl)sulfonyl)pyrrolidine; 1-(thiophen-2-ylsulfonyl)-1H-pyrazole; 1-((5-chlorothiophen-2-yl)sulfonyl)-1H-pyrazole; 1-((2-bromophenyl)sulfonyl)-1H-pyrazole; 1-(phenylsulfonyl)-1H-pyrazole; 1-((5-bromothiophen-2-yl)sulfonyl)-1H-imidazole; 1-(thiophen-2-ylsulfonyl)-1H-imidazole; 1-((5-chlorothiophen-2-yl)sulfonyl)-1H-imidazole; 1-((2-bromophenyl)sulfonyl)-1H-imidazole; 1-(phenylsulfonyl)-1H-imidazole; 1-((5-bromothiophen-2-yl)sulfonyl)-1H-pyrrole; 1-(thiophen-2-ylsulfonyl)-1H-pyrrole; 1-((5-chlorothiophen-2-yl)sulfonyl)-1H-pyrrole; 1-((2-bromophenyl)sulfonyl)-1H-pyrrole; 1-(phenylsulfonyl)-1H-pyrrole; 1-((5-bromothiophen-2-yl)sulfonyl)-1H-1,2,4-triazole; 1-(thiophen-2-ylsulfonyl)piperidine; 1-(thiophen-2-ylsulfonyl)-1H-1,2,4-triazole; 1-((5-chlorothiophen-2-yl)sulfonyl)-1H-1,2,4-triazole; 1-((2-bromophenyl)sulfonyl)-1H-1,2,4-triazole; 1-(phenylsulfonyl)-1H-1,2,4-triazole; 5-bromothiophene-2-sulfonamide; thiophene-2-sulfonamide; 5-chlorothiophene-2-sulfonamide; 1-((5-bromothiophen-2-yl)sulfonyl)piperidine; 1-((5-chlorothiophen-2-yl)sulfonyl)piperidine; 5-bromo-N-phenylthiophene-2-sulfonamide; N-phenylthiophene-2-sulfonamide; 5-chloro-N-phenylthiophene-2-sulfonamide; 1-((5-bromothiophen-2-yl)sulfonyl)-3-methyl-1H-pyrazole; 3-methyl-1-(thiophen-2-ylsulfonyl)-1H-pyrazole; 1-((5-chlorothiophen-2-yl)sulfonyl)-3-methyl-1H-pyrazole; 1-((5-bromothiophen-2-yl)sulfonyl)-3,5-dimethyl-1H-pyrazole; 3,5-dimethyl-1-(thiophen-2-ylsulfonyl)-1H-pyrazole; 1-((5-chlorothiophen-2-yl)sulfonyl)-3,5-dimethyl-1H-pyrazole; 1-((5-bromothiophen-2-yl)sulfonyl)-3-phenyl-1H-pyrazole; 3-phenyl-1-(thiophen-2-ylsulfonyl)-1H-pyrazole; and, 1-((5-chlorothiophen-2-yl)sulfonyl)-3-phenyl-1H-pyrazole, or an acceptable salt thereof.

Clause 17. A compound selected from the group consisting of: (1H-pyrazol-1-yl)(thiophen-2-yl)methanone; furan-2-yl(1H-pyrazol-1-yl)methanone; (1H-pyrrol-1-yl)(thiophen-2-yl)methanone; furan-2-yl(1H-pyrrol-1-yl)methanone; 4-(4-phenylthiazol-2-yl)-2-(trifluoromethyl)morpholine; (4-(benzo[d][1,3]dioxol-5-ylmethyl)piperazin-1-yl)(5-((4-chlorophenoxy)methyl)furan-2-yl)methanone; (1H-imidazol-1-yl)(thiophen-2-yl)methanone; furan-2-yl(1H-imidazol-1-yl)methanone; 5-bromo-N′-((5-bromothiophen-2-yl)sulfonyl)thiophene-2-sulfonohydrazide; N′-(thiophen-2-ylsulfonyl)thiophene-2-sulfonohydrazide; and, 5-chloro-N′-((5-chlorothiophen-2-yl)sulfonyl)thiophene-2-sulfonohydrazide, or an acceptable salt thereof.

Clause 18. A composition comprising a compound according to clause 17 and an acceptable carrier.

Clause 19. A method to inhibit quorum sensing in Vibrio bacteria, the method comprising: contacting the bacteria or an environment containing the bacteria with a compound that inhibits LuxR activation and/or repression but does not affect growth of the Vibrio bacteria.

Clause 20. A method to inhibit quorum sensing in Vibrio bacteria according to clause 19, where the compound that that inhibits LuxR activation and/or repression but does not affect growth of the Vibrio bacteria is a sulfonamide.

Clause 21. A method to treat a Vibrio infection in a subject in need thereof, the method comprising the step of administering to the subject a compound of Formula (I)

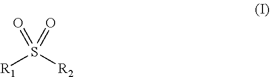

where R₁ and R₂ are independently selected from: an amino, an azide, a phenylamino, an unsubstituted or an optionally substituted aryl, an unsubstituted or an optionally substituted heterocyclic ring, or an acceptable salt.

Clause 22. A method to treat or to decrease a probability for developing a Vibrio species infection in an aquatic animal in need thereof, the method comprising: administering to the aquatic animal a therapeutically effective amount of a compound of Formula (I)

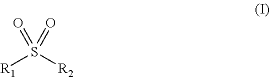

where R₁ and R₂ are independently selected from: an amino, an azide, a phenylamino, an unsubstituted or an optionally substituted aryl, an unsubstituted or an optionally substituted heterocyclic ring, or an acceptable salt.

Clause 23. A method according to clause 22, where the aquatic animal exhibits symptoms of a Vibrio species infection before the administering step.

Clause 24. A method of clause 22, where the administering comprises providing the composition to the aquaculture environment before the aquatic animal shows symptoms of Vibrio species infections.

Clause 25. A method according to clause 22, where the aquatic animal is in an aquaculture environment.

Clause 26. A method according to clause 22, where the administering comprises providing the compound to the aquaculture environment.

Clause 27. A method according to clause 22, where the administering comprises injecting the fish with the composition.

Clause 28. A method according to clause 22, where the compound is not [1-(5-bromothiophene-2-sulfonyl)-1H-pyrazole.

Clause 29. A method according to clause 22, where the compound is selected from the group consisting of: 1-((5-bromothiophen-2-yl)sulfonyl)pyrrolidine; 1-(thiophen-2-ylsulfonyl)-1H-pyrazole; 1-((5-chlorothiophen-2-yl)sulfonyl)-1H-pyrazole; 1-((2-bromophenyl)sulfonyl)-1H-pyrazole; 1-(phenylsulfonyl)-1H-pyrazole; 1-((5-bromothiophen-2-yl)sulfonyl)-1H-imidazole; 1-(thiophen-2-ylsulfonyl)-1H-imidazole; 1-((5-chlorothiophen-2-yl)sulfonyl)-1H-imidazole; 1-((2-bromophenyl)sulfonyl)-1H-imidazole; 1-(phenylsulfonyl)-1H-imidazole; 1-((5-bromothiophen-2-yl)sulfonyl)-1H-pyrrole; 1-(thiophen-2-ylsulfonyl)-1H-pyrrole; 1-((5-chlorothiophen-2-yl)sulfonyl)-1H-pyrrole; 1-((2-bromophenyl)sulfonyl)-1H-pyrrole; 1-(phenylsulfonyl)-1H-pyrrole; 1-((5-bromothiophen-2-yl)sulfonyl)-1H-1,2,4-triazole; 1-(thiophen-2-ylsulfonyl)piperidine; 1-(thiophen-2-ylsulfonyl)-1H-1,2,4-triazole; 1-((5-chlorothiophen-2-yl)sulfonyl)-1H-1,2,4-triazole; 1-((2-bromophenyl)sulfonyl)-1H-1,2,4-triazole; 1-(phenylsulfonyl)-1H-1,2,4-triazole; 5-bromothiophene-2-sulfonamide; thiophene-2-sulfonamide; 5-chlorothiophene-2-sulfonamide; 1-((5-bromothiophen-2-yl)sulfonyl)piperidine; 1-((5-chlorothiophen-2-yl)sulfonyl)piperidine; 5-bromo-N-phenylthiophene-2-sulfonamide; N-phenylthiophene-2-sulfonamide; 5-chloro-N-phenylthiophene-2-sulfonamide; 1-((5-bromothiophen-2-yl)sulfonyl)-3-methyl-1H-pyrazole; 3-methyl-1-(thiophen-2-ylsulfonyl)-1H-pyrazole; 1-((5-chlorothiophen-2-yl)sulfonyl)-3-methyl-1H-pyrazole; 1-((5-bromothiophen-2-yl)sulfonyl)-3,5-dimethyl-1H-pyrazole; 3,5-dimethyl-1-(thiophen-2-ylsulfonyl)-1H-pyrazole; 1-((5-chlorothiophen-2-yl)sulfonyl)-3,5-dimethyl-1H-pyrazole; 1-((5-bromothiophen-2-yl)sulfonyl)-3-phenyl-1H-pyrazole; 3-phenyl-1-(thiophen-2-ylsulfonyl)-1H-pyrazole; 1-((5-chlorothiophen-2-yl)sulfonyl)-3-phenyl-1H-pyrazole; 5-bromothiophene-2-sulfonyl azide; thiophene-2-sulfonyl azide; and, 5-chlorothiophene-2-sulfonyl azide, or an acceptable salt thereof.

Clause 30. A method to treat or to decrease a probability for developing a Vibrio species infection in an aquatic animal in need thereof, the method comprising: administering to the aquatic animal a therapeutically effective amount of a compound selected from the group consisting of: (1H-pyrazol-1-yl)(thiophen-2-yl)methanone; furan-2-yl(1H-pyrazol-1-yl)methanone; (1H-pyrrol-1-yl)(thiophen-2-yl)methanone; furan-2-yl(1H-pyrrol-1-yl)methanone; 4-(4-phenylthiazol-2-yl)-2-(trifluoromethyl)morpholine; (4-(benzo[d][1,3]dioxol-5-ylmethyl)piperazin-1-yl)(5-((4-chlorophenoxy)methyl)furan-2-yl)methanone; (1H-imidazol-1-yl)(thiophen-2-yl)methanone; furan-2-yl(1H-imidazol-1-yl)methanone; 5-bromo-N′-((5-bromothiophen-2-yl)sulfonyl)thiophene-2-sulfonohydrazide; N′-(thiophen-2-ylsulfonyl)thiophene-2-sulfonohydrazide; and, 5-chloro-N′-((5-chlorothiophen-2-yl)sulfonyl)thiophene-2-sulfonohydrazide, or an acceptable salt thereof.

Some embodiments of the invention are further described in paragraphs [0047] to [0093]

1. A compound of Formula (I)

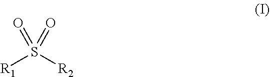

wherein R₁ and R₂ are independently selected from:
   an amino, an azide, a phenylamino, an unsubstituted or an optionally substituted aryl, an unsubstituted or an optionally substituted heterocyclic ring, or an acceptable salt thereof.

2. The compound according to claim 1, wherein R₁ is not 1H-pyrazol-1-yl when R₂ is 5-bromothiphen-2-yl.

3. A compound of Formula (I)

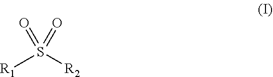

wherein R₁ and R₂ are independently selected from:

a substituted triazole or an unsubstituted triazole, a substituted pyrazole or an unsubstituted pyrazole, a substituted phenyl or an unsubstituted phenyl, a substituted thiophene, or an un-substituted thiophene,

wherein the substituted triazole, the substituted pyrazole the substituted phenyl, or the substituted thiophene are independently substituted with a methyl, an ethyl, a phenyl, a substituted phenyl, or a halogen; and

wherein R₁ is not 1H-pyrazol-1-yl when R₂ is 5-bromothiphen-2-yl.

4. A compound selected from the group consisting of: (1H-pyrazol-1-yl)(thiophen-2-yl)methanone; furan-2-yl(1H-pyrazol-1-yl)methanone; (1H-pyrrol-1-yl)(thiophen-2-yl)methanone; furan-2-yl(1H-pyrrol-1-yl)methanone; 4-(4-phenylthiazol-2-yl)-2-(trifluoromethyl)morpholine; (4-(benzo[d][1,3]dioxol-5-ylmethyl)piperazin-1-yl)(5-((4-chlorophenoxy)methyl)furan-2-yl)methanone; (1H-imidazol-1-yl)(thiophen-2-yl)methanone; 5-bromo-N′-((5-bromothiophen-2-yl)sulfonyl)thiophene-2-sulfonohydrazide; N′-(thiophen-2-ylsulfonyl)thiophene-2-sulfonohydrazide; and, 5-chloro-N′-((5-chlorothiophen-2-yl)sulfonyl)thiophene-2-sulfonohydrazide; and, furan-2-yl(1H-imidazol-1-yl)methanone, or an acceptable salt thereof.

5. A compound selected from the group consisting of: 1-(thiophen-2-ylsulfonyl)-1H-pyrazole; 1-((5-chlorothiophen-2-yl)sulfonyl)-1H-pyrazole; 1-((2-bromophenyl)sulfonyl)-1H-pyrazole; 1-(phenylsulfonyl)-1H-pyrazole; 1-((5-methylthiophen-2-yl)sulfonyl)-1H-pyrazole; 1-((2-bromophenyl)sulfonyl)-1H-pyrrole; 1-((5-bromothiophen-2-yl)sulfonyl)-1H-pyrrole; 1-(thiophen-2-ylsulfonyl)-1H-pyrrole; 1-((5-chlorothiophen-2-yl)sulfonyl)-1H-pyrrole; 1-((5-methylthiophen-2-yl)sulfonyl)-1H-pyrrole; 1-((5-bromothiophen-2-yl)sulfonyl)-1H-1,2,4-triazole; 1-((5-chlorothiophen-2-yl)sulfonyl)-1H-1,2,4-triazole; 1-((2-bromophenyl)sulfonyl)-1H-1,2,4-triazole; 1-(phenylsulfonyl)-1H-1,2,4-triazole; 1-((5-methylthiophen-2-yl)sulfonyl)-1H-1,2,4-triazole; 1-((5-bromothiophen-2-yl)sulfonyl)-3-methyl-1H-pyrazole; 3-methyl-1-(thiophen-2-ylsulfonyl)-1H-pyrazole; 1-((5-chlorothiophen-2-yl)sulfonyl)-3-methyl-1H-pyrazole; 1-((5-bromothiophen-2-yl)sulfonyl)-3-phenyl-1H-pyrazole; 3-phenyl-1-(thiophen-2-ylsulfonyl)-1H-pyrazole; 1-((5-chlorothiophen-2-yl)sulfonyl)-3-phenyl-1H-pyrazole; and 1-((5-methylthiophen-2-yl)sulfonyl)-3-phenyl-1H-pyrazole, or an acceptable salt thereof.

6. A compound selected from the group consisting of: 1-((5-methylthiophen-2-yl)sulfonyl)-1H-pyrrole; 1-(phenylsulfonyl)-1H-1,2,4-triazole; 1-((5-methylthiophen-2-yl)sulfonyl)-1H-1,2,4-triazole; and 1-((5-bromothiophen-2-yl)sulfonyl)-3-methyl-1H-pyrazole or an acceptable salt thereof.

7. A compounds selected from the group consisting of: 1-((5-chlorothiophen-2-yl)sulfonyl)-1H-pyrazole; 1-((5-bromothiophen-2-yl)sulfonyl)-3-phenyl-1H-pyrazole; 3-phenyl-1-(thiophen-2-ylsulfonyl)-1H-pyrazole; 1-((5-chlorothiophen-2-yl)sulfonyl)-3-phenyl-1H-pyrazole; 1-((5-methylthiophen-2-yl)sulfonyl)-3-phenyl-1H-pyrazole, or an acceptable salt thereof.

8. A compound comprising 3-phenyl-1-(thiophen-2-ylsulfonyl)-1H-pyrazole, or an acceptable salt thereof.

9. The compounds according to any of claims 1 to 8, wherein one or more of the compounds inhibit quorum sensing in at least one species of Vibrio.

10. The compounds according to claim 9, wherein the at least one species of Vibrio is selected from the group consisting of: Vibrio vulnificus, Vibrio parahaemolyticus, and V. campbellii.

11. The compound according to any of claims 6 to 7, wherein the compound that inhibits quorum sensing is active at concentrations of 100 μM or less.

12. The compound according to any of claims 6 to 7, wherein the compound that inhibits quorum sensing is active at concentrations of 1 μM or less.

13. The method according to any of claims 9 to 12, wherein the compound that inhibits quorum sensing inhibits LuxR activation and/or repression but does not affect growth of the Vibrio bacteria.

14. A method to inhibit quorum sensing in Vibrio bacteria, the method comprising the step of:

contacting at least one species of Vibrio bacteria or an environment that includes at least one species of Vibrio bacteria with a compound that inhibits quorum sensing.

15. The method according to claim 14, wherein the compound that inhibits quorum sensing inhibits LuxR activation and/or repression but does not affect growth of the Vibrio bacteria.

16. The method according to any of claims 14 to 15, wherein the compound that inhibits quorum sensing is a compound of Formula (I)

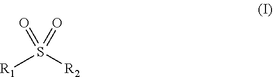

wherein R₁ and R₂ are independently selected from:

an amino, an azide, a phenylamino, an unsubstituted or an optionally substituted aryl, an unsubstituted or an optionally substituted heterocyclic ring, or an acceptable salt thereof.

17. The method according to any of claims 14 to 15, wherein the compound that inhibits quorum sensing is a compound of Formula (I)

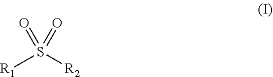

wherein R₁ and R₂ are independently selected from:

an amino, an azide, a phenylamino, an unsubstituted or an optionally substituted aryl, an unsubstituted or an optionally substituted heterocyclic ring, or an acceptable salt thereof; and wherein R₁ is not 1H-pyrazol-1-yl when R₂ is 5-bromothiphen-2-yl.

18. The method according to any of claims 14 to 15, wherein the compound that inhibits quorum sensing is a compound of Formula (II)

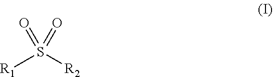

wherein R₁ and R₂ are independently selected from:

a substituted triazole or an unsubstituted triazole, a substituted pyrazole or an unsubstituted pyrazole, a substituted phenyl or an unsubstituted phenyl, a substituted thiophene, or an unsubstituted thiophene,

wherein the substituted triazole, the substituted pyrazole, the substituted phenyl or the substituted thiophene are independently substituted with a methyl, an ethyl, a phenyl, a substitute phenyl, or a halogen; and

wherein, R₁ is not 1H-pyrazol-1-yl when R₂ is 5-bromothiphen-2-yl.

19. The method according to any of claims 14 to 15, wherein the compound that inhibits quorum sensing is a compound selected from the group consisting of: (1H-pyrazol-1-yl)(thiophen-2-yl)methanone; furan-2-yl(1H-pyrazol-1-yl)methanone; (1H-pyrrol-1-yl)(thiophen-2-yl)methanone; furan-2-yl(1H-pyrrol-1-yl)methanone; 4-(4-phenylthiazol-2-yl)-2-(trifluoromethyl)morpholine; (4-(benzo[d][1,3]dioxol-5-ylmethyl)piperazin-1-yl)(5-((4-chlorophenoxy)methyl)furan-2-yl)methanone; (1H-imidazol-1-yl)(thiophen-2-yl)methanone; 5-bromo-N′-((5-bromothiophen-2-yl)sulfonyl)thiophene-2-sulfonohydrazide; N′-(thiophen-2-ylsulfonyl)thiophene-2-sulfonohydrazide; and, 5-chloro-N′-((5-chlorothiophen-2-yl)sulfonyl)thiophene-2-sulfonohydrazide; and, furan-2-yl(1H-imidazol-1-yl)methanone, or an acceptable salt thereof.

20. The method according to any of claims 14 to 15, wherein the compound that inhibits quorum sensing is a compound selected from the group consisting of: 1-(thiophen-2-ylsulfonyl)-1H-pyrazole; 1-((5-chlorothiophen-2-yl)sulfonyl)-1H-pyrazole; 1-((2-bromophenyl)sulfonyl)-1H-pyrazole; 1-(phenylsulfonyl)-1H-pyrazole; 1-((5-methylthiophen-2-yl)sulfonyl)-1H-pyrazole; 1-((2-bromophenyl)sulfonyl)-1H-pyrrole; 1-((5-bromothiophen-2-yl)sulfonyl)-1H-pyrrole; 1-(thiophen-2-ylsulfonyl)-1H-pyrrole; 1-((5-chlorothiophen-2-yl)sulfonyl)-1H-pyrrole; 1-((5-methylthiophen-2-yl)sulfonyl)-1H-pyrrole; 1-((5-bromothiophen-2-yl)sulfonyl)-1H-1,2,4-triazole; 1-((5-chlorothiophen-2-yl)sulfonyl)-1H-1,2,4-triazole; 1-((2-bromophenyl)sulfonyl)-1H-1,2,4-triazole; 1-(phenylsulfonyl)-1H-1,2,4-triazole; 1-((5-methylthiophen-2-yl)sulfonyl)-1H-1,2,4-triazole; 1-((5-bromothiophen-2-yl)sulfonyl)-3-methyl-1H-pyrazole; 3-methyl-1-(thiophen-2-ylsulfonyl)-1H-pyrazole; 1-((5-chlorothiophen-2-yl)sulfonyl)-3-methyl-1H-pyrazole; 1-((5-bromothiophen-2-yl)sulfonyl)-3-phenyl-1H-pyrazole; 3-phenyl-1-(thiophen-2-ylsulfonyl)-1H-pyrazole; 1-((5-chlorothiophen-2-yl)sulfonyl)-3-phenyl-1H-pyrazole; and 1-((5-methylthiophen-2-yl)sulfonyl)-3-phenyl-1H-pyrazole, or an acceptable salt thereof.

21. The method according to any of claims 14 to 15, wherein the compound that inhibits quorum sensing is a compound selected from the group consisting of: 1-((5-methylthiophen-2-yl)sulfonyl)-1H-pyrrole; 1-(phenylsulfonyl)-1H-1,2,4-triazole; 1-((5-methylthiophen-2-yl)sulfonyl)-1H-1,2,4-triazole; and 1-((5-bromothiophen-2-yl)sulfonyl)-3-methyl-1H-pyrazole or an acceptable salt thereof.

22. A compounds selected from the group consisting of: 1-((5-chlorothiophen-2-yl)sulfonyl)-1H-pyrazole; 1-((5-bromothiophen-2-yl)sulfonyl)-3-phenyl-1H-pyrazole; 3-phenyl-1-(thiophen-2-ylsulfonyl)-1H-pyrazole; 1-((5-chlorothiophen-2-yl)sulfonyl)-3-phenyl-1H-pyrazole; 1-((5-methylthiophen-2-yl)sulfonyl)-3-phenyl-1H-pyrazole, or an acceptable salt thereof.

23. The method according to any of claims 14 to 15, wherein the compound that inhibits quorum sensing is a compound comprising 3-phenyl-1-(thiophen-2-ylsulfonyl)-1H-pyrazole, or an acceptable salt thereof.

24. The method according to any of claims 14 to 23, wherein the compound inhibits quorum sensing in at least one species of Vibrio.

25. The method according to any of claims 14 to 23, wherein at least one species of Vibrio is selected from the group consisting of: Vibrio vulnificus, Vibrio parahaemolyticus, and V. campbellii.

26. The method according to any of claims 14 to 25, wherein the compound that inhibits quorum sensing is active at concentrations of 100 μM or less.

27. The method according to any of claims 14 to 25, wherein the compound that inhibits quorum sensing is active at concentrations of 1 μM or less.

28. The method according to any of claims 14 to 27, further including the step of adding the compound to water used in aquaculture.

29. A method to treat or to reduce the risk for developing a Vibrio infection in a subject in need thereof, the method comprising the step of:

administering at least one therapeutically effective dose of a compound that inhibits quorum sensing in Vibrio bacteria or a pharmaceutically acceptable salt of the compound, to a patient diagnosed with or at risk for developing an infection with at least one species of Vibrio bacteria.

30. The method according to claim 29, wherein the compound that inhibits quorum sensing in Vibrio bacteria inhibits LuxR activation and/or repression but does not affect growth of the Vibrio bacteria.

31. The method according to any of claims 29 to 30, wherein the compound that inhibits quorum sensing is a compound of Formula (I)

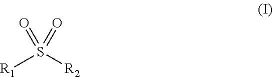

wherein R₁ and R₂ are independently selected from:

an amino, an azide, a phenylamino, an unsubstituted or an optionally substituted aryl, an unsubstituted or an optionally substituted heterocyclic ring, or an acceptable salt thereof.

32. The method according to any of claims 29 to 30, wherein the compound that inhibits quorum sensing is a compound of Formula (I)

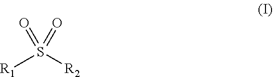

wherein R₁ and R₂ are independently selected from:

an amino, an azide, a phenylamino, an unsubstituted or an optionally substituted aryl, an unsubstituted or an optionally substituted heterocyclic ring, or an acceptable salt thereof; and

wherein R₁ is not 1H-pyrazol-1-yl when R₂ is 5-bromothiphen-2-yl.

33. The method according to any of claims 29 to 30, wherein the compound that inhibits quorum sensing is a compound of Formula (I)

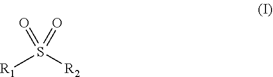

wherein R₁ and R₂ are independently selected from:

a substituted triazole or an unsubstituted triazole, a substituted pyrazole or an unsubstituted pyrazole, a substituted phenyl or an unsubstituted phenyl, a substituted thiophene or an unsubstituted thiophene,

wherein the substituted triazole, the substituted pyrazole, the substituted phenyl or the substituted thiophene are independently substituted with a methyl, an ethyl, a phenyl, a substitute phenyl, or a halogen; and

wherein, R₁ is not 1H-pyrazol-1-yl when R₂ is 5-bromothiphen-2-yl.

34. The method according to any of claims 29 to 30, wherein the compound that inhibits quorum sensing is a compound selected from the group consisting of: (1H-pyrazol-1-yl)(thiophen-2-yl)methanone; furan-2-yl(1H-pyrazol-1-yl)methanone; (1H-pyrrol-1-yl)(thiophen-2-yl)methanone; furan-2-yl(1H-pyrrol-1-yl)methanone; 4-(4-phenylthiazol-2-yl)-2-(trifluoromethyl)morpholine; (4-(benzo[d][1,3]dioxol-5-ylmethyl)piperazin-1-yl)(5-((4-chlorophenoxy)methyl)furan-2-yl)methanone; (1H-imidazol-1-yl)(thiophen-2-yl)methanone; 5-bromo-N′-((5-bromothiophen-2-yl)sulfonyl)thiophene-2-sulfonohydrazide; N′-(thiophen-2-ylsulfonyl)thiophene-2-sulfonohydrazide; and, 5-chloro-N′-((5-chlorothiophen-2-yl)sulfonyl)thiophene-2-sulfonohydrazide; and, furan-2-yl(1H-imidazol-1-yl)methanone, or an acceptable salt thereof.

35. The method according to any of claims 29 to 30, wherein the compound that inhibits quorum sensing is a compound selected from the group consisting of: 1-(thiophen-2-ylsulfonyl)-1H-pyrazole; 1-((5-chlorothiophen-2-yl)sulfonyl)-1H-pyrazole; 1-((2-bromophenyl)sulfonyl)-1H-pyrazole; 1-(phenylsulfonyl)-1H-pyrazole; 1-((5-methylthiophen-2-yl)sulfonyl)-1H-pyrazole; 1-((2-bromophenyl)sulfonyl)-1H-pyrrole; 1-((5-bromothiophen-2-yl)sulfonyl)-1H-pyrrole; 1-(thiophen-2-ylsulfonyl)-1H-pyrrole; 1-((5-chlorothiophen-2-yl)sulfonyl)-1H-pyrrole; 1-((5-methylthiophen-2-yl)sulfonyl)-1H-pyrrole; 1-((5-bromothiophen-2-yl)sulfonyl)-1H-1,2,4-triazole; 1-((5-chlorothiophen-2-yl)sulfonyl)-1H-1,2,4-triazole; 1-((2-bromophenyl)sulfonyl)-1H-1,2,4-triazole; 1-(phenylsulfonyl)-1H-1,2,4-triazole; 1-((5-methylthiophen-2-yl)sulfonyl)-1H-1,2,4-triazole; 1-((5-bromothiophen-2-yl)sulfonyl)-3-methyl-1H-pyrazole; 3-methyl-1-(thiophen-2-ylsulfonyl)-1H-pyrazole; 1-((5-chlorothiophen-2-yl)sulfonyl)-3-methyl-1H-pyrazole; 1-((5-bromothiophen-2-yl)sulfonyl)-3-phenyl-1H-pyrazole; 3-phenyl-1-(thiophen-2-ylsulfonyl)-1H-pyrazole; 1-((5-chlorothiophen-2-yl)sulfonyl)-3-phenyl-1H-pyrazole; and 1-((5-methylthiophen-2-yl)sulfonyl)-3-phenyl-1H-pyrazole, or an acceptable salt thereof.

36. The method according to any of claims 29 to 30, wherein the compound that inhibits quorum sensing is a compound selected from the group consisting of: 1-((5-methylthiophen-2-yl)sulfonyl)-1H-pyrrole; 1-(phenylsulfonyl)-1H-1,2,4-triazole; 1-((5-methylthiophen-2-yl)sulfonyl)-1H-1,2,4-triazole; and 1-((5-bromothiophen-2-yl)sulfonyl)-3-methyl-1H-pyrazole or an acceptable salt thereof.

37. The method according to any of claims 29 to 30, wherein the compound that inhibits quorum sensing is a compounds selected from the group consisting of: 1-((5-chlorothiophen-2-yl)sulfonyl)-1H-pyrazole; 1-((5-bromothiophen-2-yl)sulfonyl)-3-phenyl-1H-pyrazole; 3-phenyl-1-(thiophen-2-ylsulfonyl)-1H-pyrazole; 1-((5-chlorothiophen-2-yl)sulfonyl)-3-phenyl-1H-pyrazole; 1-((5-methylthiophen-2-yl)sulfonyl)-3-phenyl-1H-pyrazole, or an acceptable salt thereof.

38. The method according to any of claims 29 to 30, wherein the compound that inhibits quorum sensing is a compound comprising 3-phenyl-1-(thiophen-2-ylsulfonyl)-1H-pyrazole, or an acceptable salt thereof.

39. The method according to any of claims 29 to 38, wherein the compound inhibits quorum sensing in at least one species of Vibrio.

40. The method according to any of claims 29 to 38, wherein at least one species of Vibrio is selected from the group consisting of: Vibrio vulnificus, Vibrio parahaemolyticus, and V. campbellii.

41. The method according to any of claims 29 to 40, wherein the at least one therapeutically effective dose of the compound includes from about 1 pg/kg to about 10 μg/kg, from about 1 pg/kg to about 1 μg/kg, from about 100 pg/kg to about 500 ng/kg, from about 1 pg/kg to about 1 ng/kg, from about 1 pg/kg to about 500 pg/kg, from about 100 pg/kg to about 500 ng/kg, from about 100 pg/kg to about 100 ng/kg, from about 1 ng/kg to about 10 mg/kg, from about 1 ng/kg to 1 mg/kg, from about 1 ng/kg to about 1 μg/kg, from about 1 ng/kg to about 500 ng/kg, from about 100 ng/kg to about 500 μg/kg, from about 100 ng/kg to about 100 μg/kg, from about 1 μg/kg to about 500 μg/kg, or from about 1 μg/kg to about 100 μg/kg.

42. The method according to claim 41, wherein the dose/kg refers to the dose per kilogram of a patient's body mass or body weight.

43. The method according to claims 29 to 42, wherein the patient is a fish.

44. The method according to any of claims 29 to 43, wherein the compound is added to fish feed formulations.

45. The method according to claim 44 wherein the fish feed comprises from about 1 to about 2500 mg of the compound or a physiologically acceptable derivative or salt thereof in association with and per kg of the fish feed composition.

### BRIEF DESCRIPTION OF THE FIGURES

FIG. 1. The quorum sensing pathway in Vibrio species. Autoinducer molecules are bound by membrane-bound histidine kinase receptors, which alters the phosphorylation cascade downstream. At high cell densities, the production of the transcription factor LuxR is maximal, and LuxR activates and represses genes encoding proteins with various functions, some of which are listed in the diagram. LuxR protein homologs in various Vibrio species are listed.

FIG. 2A. Screen for LuxR inhibitors in Escherichia coli bioassay. Diagram of the two plasmids in the E. coli bioassay used to screen for LuxR inhibitors. Plasmid 1 (pJV064) contains divergent promoters for V. campbellii luxCDABE and 05222 driving expression of gfp and mCherry, respectively. Plasmid 2 (pKM699) contains the V. campbellii luxR gene under control of its native promoter.

FIG. 2B. Fluorescence expression (GFP/OD600 or mCherry/OD600) in E. coli bioassay cells (with pJV064) expressing LuxR (pKM699) or the empty vector control (pLAFR2).

FIG. 2C. Structure of LuxR inhibitors identified and verified from the compound library.

FIG. 2D. Production of GFP (GFP/OD600) in the presence of LuxR inhibitors titrated into the E. coli bioassay strain (pKM699, pJV064). DMSO was titrated as a solvent control with an equal volume to the 100 μM concentration of compound and compared to cells in which nothing was added (−, plotted at 100 μM point on x-axis). Data shown represent the mean and standard deviation of at least three biological replicates.

FIG. 2F. Production of GFP (mCherry/OD₆₀₀) in the presence of LuxR inhibitors titrated into the E. coli bioassay strain (pKM699, pJV064). DMSO was titrated as a solvent control with an equal volume to the 100 μM concentration of compound and compared to cells in which nothing was added (−, plotted at 100 μM point on x-axis). Data shown represent the mean and standard deviation of at least three biological replicates.

FIG. 3A. A Panel of thiophenesulfonamide molecules that inhibit LuxR. Substrates with modifications to the heteroaromatic ring (pyrazole in the case of Qstatin) have number designations, and substrates with modifications to the thiophene ring have letter designations.

FIG. 3B. Production of GFP (GFP/OD₆₀₀) in the presence of thiophenesulfonamide compounds titrated into the E. coli bioassay strain (pKM699, pJV064). DMSO was titrated as a solvent control with an equal volume to the 100 μM concentration of compound and compared to cells in which nothing was added (−, plotted at 100 μM point on x-axis). Data shown represent the mean and standard deviation of at least three biological replicates.

FIG. 3C. Production of GFP (mCherry//OD₆₀₀) in the presence of thiophenesulfonamide compounds titrated into the E. coli bioassay strain (pKM699, pJV064). DMSO was titrated as a solvent control with an equal volume to the 100 μM concentration of compound and compared to cells in which nothing was added (−, plotted at 100 μM point on x-axis). Data shown represent the mean and standard deviation of at least three biological replicates.

FIG. 4A. Thiophenesulfonamides have a range of inhibition against Vibrio species. Titration of molecules from the top panel of thiophenesulfonamides in Vibrio strains compared to DMSO solvent control (DMSO was titrated with an equal volume to the 100 μM concentration of compound). Data shown represent the mean and standard deviation of three biological replicates.

FIG. 4B. Titration of molecules from the top panel of thiophenesulfonamides in Vibrio strains compared to DMSO solvent control (DMSO was titrated with an equal volume to the 100 μM concentration of compound). Data shown represent the mean and standard deviation of three biological replicates.

FIG. 4C. Titration of molecules from the top panel of thiophenesulfonamides in Vibrio strains compared to DMSO solvent control (DMSO was titrated with an equal volume to the 100 μM concentration of compound). Data shown represent the mean and standard deviation of three biological replicates.

FIG. 4D. Titration of molecules from the top panel of thiophenesulfonamides in Vibrio strains compared to DMSO solvent control (DMSO was titrated with an equal volume to the 100 μM concentration of compound). Data shown represent the mean and standard deviation of three biological replicates.

FIG. 4E. Titration of molecules from the top panel of thiophenesulfonamides in Vibrio strains compared to DMSO solvent control (DMSO was titrated with an equal volume to the 100 μM concentration of compound). Data shown represent the mean and standard deviation of three biological replicates.

FIG. 4F. Protease activity (final assay OD₄₂₀/initial culture OD₆₀₀) for wild-type and mutant Vibrio strains in the presence of 2.5 μM PTSP or an equal volume of the DMSO solvent. Asterisks indicate significant differences (Unpaired t test, two-stage step-up (Benjamini, Krieger, and Yekutieli), n=3; *, p=0.05; **, p=0.01; ***, p=0.001; ****, p=0.0001; ns, not significant).

FIG. 4G. Protease activity (final assay OD₄₂₀/initial culture OD₆₀₀) for Vibrio strains in the presence of PTSP titrated into the cultures. Data shown represent the mean and standard deviation of three biological replicates.

### DETAILED DESCRIPTION

Additional embodiments, features, and advantages of the disclosure will be apparent from the following detailed description and through practice of the disclosure.

Before the present disclosure is further described, it is to be understood that this disclosure is not limited to particular embodiments described, as such may, of course, vary. It is also to be understood that the terminology used herein is for the purpose of describing particular embodiments only, and is not intended to be limiting, since the scope of the present disclosure will be limited only by the appended claims.

Unless defined otherwise, all technical and scientific terms used herein have the same meaning as is commonly understood by one of ordinary skill in the art to which this disclosure belongs. All patents, applications, published applications and other publications referred to herein are incorporated by reference in their entireties. If a definition set forth in this section is contrary to or otherwise inconsistent with a definition set forth in a patent, application, or other publication that is herein incorporated by reference, the definition set forth in this section prevails over the definition incorporated herein by reference.

The singular forms “a,” “an,” and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation. The terms “including,” “containing,” and “comprising” are used in their open, non-limiting sense.

To provide a more concise description, some of the quantitative expressions given herein are not qualified with the term “about.” It is understood that, whether the term “about” is used explicitly or not, every quantity given herein is meant to refer to the actual given value, and it is also meant to refer to the approximation to such given value that would reasonably be inferred based on the ordinary skill in the art, including equivalents and approximations due to the experimental and/or measurement conditions for such given value. Whenever a yield is given as a percentage, such yield refers to a mass of the entity for which the yield is given with respect to the maximum amount of the same entity that could be obtained under the particular stoichiometric conditions. Concentrations that are given as percentages refer to mass ratios, unless indicated differently.

Except as otherwise noted, the methods and techniques of the present embodiments are generally performed according to conventional methods well known in the art and as described in various general and more specific references that are cited and discussed throughout the present specification. See, e.g., Loudon, Organic Chemistry, Fourth Edition, New York: Oxford University Press, 2002, pp. 360-361, 1084-1085; Smith and March, March's Advanced Organic Chemistry: Reactions, Mechanisms, and Structure, Fifth Edition, Wiley-Interscience, 2001.

Certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable subcombination. All combinations of the embodiments pertaining to the chemical groups represented by the variables are specifically embraced by the present disclosure and are disclosed herein just as if each and every combination was individually and explicitly disclosed, to the extent that such combinations embrace compounds that are stable compounds (i.e., compounds that can be isolated, characterized, and tested for biological activity). In addition, all subcombinations of the chemical groups listed in the embodiments describing such variables are also specifically embraced by the present disclosure and are disclosed herein just as if each and every such sub-combination of chemical groups was individually and explicitly disclosed herein.

“Alkyl” includes a chain of carbon atoms, which is optionally branched and contains from 1 to 20 carbon atoms. It is to be further understood that in certain embodiments, alkyl may be advantageously of limited length, including C₁-C₁₂, C₁-C₁₀, C₁-C₉, C₁-C₈, C₁-C₇, C₁-C₆, C₁-C₄, and C₁-C₃. Illustratively, such particularly limited length alkyl groups, including C₁-C₈, C₁-C₇, C₁-C₆, C₁-C₄, and C₁-C₃ and the like may be referred to as “lower alkyl.” Illustrative alkyl groups include, but are not limited to, methyl, ethyl, n-propyl, isopropyl, n-butyl, isobutyl, sec-butyl, tert-butyl, pentyl, 2-pentyl, 3-pentyl, neopentyl, hexyl, heptyl, octyl, and the like. It will be understood that “alkyl” may be combined with other groups, such as those provided above, to form a functionalized alkyl. By way of example, the combination of an “alkyl” group, as described herein, with a “carboxy” group may be referred to as a “carboxyalkyl” group. Other non-limiting examples include hydroxyalkyl, aminoalkyl, and the like.

“Hydroxy” or “hydroxyl” refers to an —OH group.
“Alkoxy” refers to both an —O-(alkyl). Representative examples include, but are not limited to, methoxy, ethoxy, propoxy, butoxy, and the like.
“Halo” or “halogen” refers to fluorine, chlorine, bromine, or iodine.
“Bond” refers to a covalent bond.

“Aryl” refers to monocyclic or polycyclic (e.g., having 2 or more fused rings). Preferred polycyclic aryl groups have 2 or 3 rings, which may be fused or not fused. Aryl groups include phenyl, naphthyl, indanyl, indenyl, anthracenyl, and phenanthrenyl among others.

“Independently” means that the subsequently described event or circumstance is to be read on its own relative to other similar events or circumstances. For example, in a circumstance where several equivalent hydrogen groups are optionally substituted by another group described in the circumstance, the use of “independently optionally” means that each instance of a hydrogen atom on the group may be substituted by another group, where the groups replacing each of the hydrogen atoms may be the same or different. Or for example, where multiple groups exist all of which can be selected from a set of possibilities, the use of “independently” means that each of the groups can be selected from the set of possibilities separate from any other group, and the groups selected in the circumstance may be the same or different.

“Substituted” means that one or more hydrogens on the designated atom is replaced with a selection from the indicated group, provided that the designated atom's normal valency under the existing circumstances is not exceeded, and that the substitution results in a stable compound. Combinations of substituents and/or variables are permissible only if such combinations result in stable compounds.

“Optionally substituted” means optional substitution with the specified groups, radicals or moieties.

“Acceptable salt” refers to those salts which counter ions which may be used in pharmaceuticals. See, generally, S. M. Berge, et al., “Pharmaceutical Salts,” J. Pharm. Sci., 1977, 66, 1-19. Preferred pharmaceutically acceptable salts are those that are pharmacologically effective and suitable for contact with the tissues of subjects without undue toxicity, irritation, or allergic response. A compound described herein may possess a sufficiently acidic group, a sufficiently basic group, both types of functional groups, or more than one of each type, and accordingly react with a number of inorganic or organic bases, and inorganic and organic acids, to form a pharmaceutically acceptable salt. Such salts include:

(1) acid addition salts, which can be obtained by reaction of the free base of the parent compound with inorganic acids such as hydrochloric acid, hydrobromic acid, nitric acid, phosphoric acid, sulfuric acid, and perchloric acid and the like, or with organic acids such as acetic acid, oxalic acid, (D) or (L) malic acid, maleic acid, methane sulfonic acid, ethanesulfonic acid, p-toluenesulfonic acid, salicylic acid, tartaric acid, citric acid, succinic acid or malonic acid and the like; or

(2) salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, trimethamine, N-methylglucamine, and the like.

Pharmaceutically acceptable salts are well known to those skilled in the art, and any such pharmaceutically acceptable salt may be contemplated in connection with the embodiments described herein. Examples of pharmaceutically acceptable salts include sulfates, pyrosulfates, bisulfates, sulfites, bisulfites, phosphates, monohydrogen-phosphates, dihydrogenphosphates, metaphosphates, pyrophosphates, chlorides, bromides, iodides, acetates, propionates, decanoates, caprylates, acrylates, formates, isobutyrates, caproates, heptanoates, propiolates, oxalates, malonates, succinates, suberates, sebacates, fumarates, maleates, butyne-1,4-dioates, hexyne-1,6-dioates, benzoates, chlorobenzoates, methylbenzoates, dinitrobenzoates, hydroxybenzoates, methoxybenzoates, phthalates, sulfonates, methylsulfonates, propylsulfonates, besylates, xylenesulfonates, naphthalene-1-sulfonates, naphthalene-2-sulfonates, phenylacetates, phenylpropionates, phenylbutyrates, citrates, lactates, γ-hydroxybutyrates, glycolates, tartrates, and mandelates. Lists of other suitable pharmaceutically acceptable salts are found in Remington's Pharmaceutical Sciences, 17th Edition, Mack Publishing Company, Easton, Pa., 1985.

For a compound of Formula (I) that contains a basic nitrogen, a pharmaceutically acceptable salt may be prepared by any suitable method available in the art, for example, treatment of the free base with an inorganic acid, such as hydrochloric acid, hydrobromic acid, sulfuric acid, sulfamic acid, nitric acid, boric acid, phosphoric acid, and the like, or with an organic acid, such as acetic acid, phenylacetic acid, propionic acid, stearic acid, lactic acid, ascorbic acid, maleic acid, hydroxymaleic acid, isethionic acid, succinic acid, valeric acid, fumaric acid, malonic acid, pyruvic acid, oxalic acid, glycolic acid, salicylic acid, oleic acid, palmitic acid, lauric acid, a pyranosidyl acid, such as glucuronic acid or galacturonic acid, an alpha-hydroxy acid, such as mandelic acid, citric acid, or tartaric acid, an amino acid, such as aspartic acid or glutamic acid, an aromatic acid, such as benzoic acid, 2-acetoxybenzoic acid, naphthoic acid, or cinnamic acid, a sulfonic acid, such as laurylsulfonic acid, p-toluenesulfonic acid, methanesulfonic acid, or ethanesulfonic acid, or any compatible mixture of acids such as those given as examples herein, and any other acid and mixture thereof that are regarded as equivalents or acceptable substitutes in light of the ordinary level of skill in this technology.

The disclosure also relates to pharmaceutically acceptable prodrugs of the compounds of Formula (I) and to treatment methods employing such pharmaceutically acceptable prodrugs. The term “prodrug” means a precursor of a designated compound that, following administration to a subject, yields the compound in vivo via a chemical or physiological process such as solvolysis or enzymatic cleavage, or under physiological conditions (e.g., a prodrug on being brought to physiological pH is converted to the compound of Formula (I). A “pharmaceutically acceptable prodrug” is a prodrug that is non-toxic, biologically tolerable, and otherwise biologically suitable for administration to the subject. Illustrative procedures for the selection and preparation of suitable prodrug derivatives are described, for example, in “Design of Prodrugs”, ed. H. Bundgaard, Elsevier, 1985.

The present disclosure also relates to pharmaceutically active metabolites of compounds of Formula (I) and to uses of such metabolites in the methods of the disclosure. A “pharmaceutically active metabolite” means a pharmacologically active product of metabolism in the body of a compound of Formula (I), or salt thereof. Prodrugs and active metabolites of a compound may be determined using routine techniques known or available in the art. See, e.g., Bertolini et al., J. Med. Chem. 1997, 40, 2011-2016; Shan et al., J. Pharm. Sci. 1997, 86 (7), 765-767; Bagshawe, Drug Dev. Res. 1995, 34, 220-230; Bodor, Adv. Drug Res. 1984, 13, 255-331; Bundgaard, Design of Prodrugs (Elsevier Press, 1985); and Larsen, Design and Application of Prodrugs, Drug Design and Development (Krogsgaard-Larsen et al., eds., Harwood Academic Publishers, 1991).

Any formula depicted herein is intended to represent a compound of that structural formula as well as certain variations or forms. For example, a formula given herein is intended to include a racemic form, or one or more enantiomeric, diastereomeric, or geometric isomers, or a mixture thereof. Additionally, any formula given herein is intended to refer also to a hydrate, solvate, or polymorph of such a compound, or a mixture thereof

Although the present invention contemplates all individual enantiomers and diastereomers, as well as mixtures of the enantiomers of the compounds, including racemates, the compounds with the absolute configuration as set forth below are particularly preferred.

Individual isomers, enantiomers, and diastereomers may be separated or resolved by one of ordinary skill in the art at any convenient point in the synthesis of compounds of the invention, by methods such as selective crystallization techniques or chiral chromatography (See for example, J. Jacques, et al., “Enantiomers, Racemates, and Resolutions”, John Wiley and Sons, Inc., 1981, and E. L. Eliel and S. H. Wilen, “Stereochemistry of Organic Compounds”, Wiley-Interscience, 1994).

Any formula given herein is also intended to represent unlabeled forms as well as isotopically labeled forms of the compounds. Isotopically labeled compounds have structures depicted by the formulas given herein except that one or more atoms are replaced by an atom having a selected atomic mass or mass number. Examples of isotopes that can be incorporated into compounds of the disclosure include isotopes of hydrogen, carbon, nitrogen, oxygen, phosphorous, fluorine, chlorine, and iodine, such as ²H, ³H, ¹¹C, ¹³C, ¹⁴C, ¹⁵N, ¹⁸O, ¹⁷O, ³¹P, ³²P, ³⁵S, ¹⁸F, ³⁶Cl, and ¹²⁵I, respectively. Such isotopically labelled compounds are useful in metabolic studies (preferably with ¹⁴C), reaction kinetic studies (with, for example ²H or ³H), detection or imaging techniques [such as positron emission tomography (PET) or single-photon emission computed tomography (SPECT)] including drug or substrate tissue distribution assays, or in radioactive treatment of patients. Further, substitution with heavier isotopes such as deuterium (i.e., ²H) may afford certain therapeutic advantages resulting from greater metabolic stability, for example increased in vivo half-life or reduced dosage requirements. Isotopically labeled compounds of this disclosure and prodrugs thereof can generally be prepared by carrying out the procedures disclosed in the schemes or in the examples and preparations described below by substituting a readily available isotopically labeled reagent for a non-isotopically labeled reagent.

Any disubstituent referred to herein is meant to encompass the various attachment possibilities when more than one of such possibilities are allowed. For example, reference to disubstituent -A-B-, where A #B, refers herein to such disubstituent with A attached to a first substituted member and B attached to a second substituted member, and it also refers to such disubstituent with A attached to the second substituted member and B attached to the first substituted member.

Unless explicated stated or clearly implied, the terms ‘inhibit’ and/or “inhibits’ include both complete inhibition and partial repression for example: 100%, 95%, 90%, 85%, 80%, 75%, or 70%, reduction in an observed and/or measured parameter such as quorum sensing constitutes inhibition of quorum sensing.

“Aquaculture” refers to the cultivation, breeding, raising, production, propagation and/or harvesting of an aquatic or marine animal, generally in an aquaculture environment or artificial environment such as a tank (e.g., an aquarium), a raceway, a tidal basin, a pond, a pool, a paddy, a lake, etc., or in an enclosed or fenced off portion of the animals natural habitat, such as a pond, a pool, a paddy, a lake, an estuary, an ocean, a marsh (e.g., a tidal marsh), a lagoon (e.g., a tidal lagoon), etc.

“Aquatic animal” refers to organisms that live in an aquatic or marine environment. Non-limiting examples of aquatic animals or aquaculture species are provided. In some embodiments, the aquaculture species may include, but are not limited to, aquatic species present, either fully or partially, in an aquatic environment, such as one or more of aquaculture fish and invertebrates. In some embodiments, the aquatic animal is a fish or a mollusk. In other embodiments, the aquatic animal is coral. Aquatic animals or aquaculture species may be raised for consumption, ornamental uses, or for other reasons. The fish may be any fish, with exemplary particular species including shrimp, such as Whiteleg shrimp or Penaeus vannamei, Tiger shrimp, etc.; tilapia, such as Nile tilapia, blue tilapia, Mozambique tilapia, tilapiine cichlids, or hybrids thereof; sea bream, such as sheepshead, scup, yellowfin bream, gilt-head bream, Saucereye porgies, red sea bream, or hybrids thereof; carp, such as goldfish, koi, common carp, Asian carp, Indian carp, black carp, grass carp, silver carp, bighead carp, major carp, rohu, or hybrids thereof; baitfish; clownfish; salmon, such as pink salmon, chum salmon, sockeye salmon, coho salmon, Atlantic salmon, chinook salmon, masu salmon or hybrids thereof; trout, such as rainbow trout, Adriatic trout, Bonneville cutthroat trout, brook trout, steelhead trout or hybrids thereof; cod, such as Atlantic northeast cod, Atlantic northwest cod, Pacific cod, or hybrids thereof; halibut, such as Pacific halibut, Atlantic halibut, or hybrids thereof; snapper, such as red snapper, bluefish or hybrids thereof. The composition and/or combination may be provided to any crustacean, including, but not limited to, shrimp, such as Chinese white shrimp, pink shrimp, black tiger shrimp, freshwater shrimp, gulf shrimp, Pacific white shrimp, whiteleg shrimp, giant tiger shrimp, rock shrimp, Akiama paste shrimp, Southern rough shrimp, fleshy prawn, banana prawn, Northern prawn, or hybrids thereof; crab, such as blue crab, peekytoe crab, spanner crab, Jonah crab, snow crab, king crab, stone crab, Dungeness crab, soft-shell crab, Cromer crab, or hybrids thereof; lobster, such as American lobster, spiny lobster, squat lobster, or hybrids thereof; crayfish or crawfish; krill; copepods; barnacles, such as goose barnacle, picoroco barnacle, or hybrids thereof. In other embodiments, the crustacean is selected from shrimp, crab, lobster, crayfish, hill, copepods, barnacles, or hybrids thereof. The mollusk may be selected from squid, such as common squid, Patagonian squid, longfin inshore squid, neon flying squid, Argentine shortfin squid, Humboldt squid, Japanese flying squid, Wellington squid, or hybrids thereof; octopus, such as the common octopus; clams, such as hard clam, soft-shell clam, ocean quahog, surf clam, Asari, Hamaguri, Vongola, Cozza, Tellina, or hybrids thereof; oysters, such as Pacific oyster, rock oyster, European flat oyster, Portuguese oyster, or hybrids thereof; mussel, such as blue mussel, freshwater mussel, green-lipped mussel, Asian green mussel, Mediterranean mussel, Baltic mussel, or hybrids thereof; abalone; conchs; rock snails; whelks; cockles; or combinations thereof.

The term “subject” refers to a mammalian patient in need of such treatment, such as a human.

Feed compositions comprising the compounds described herein can be administered to any aquatic animal. In some embodiments, the feed compositions are administered to aquatic animals in connection with the farming of, for example, fish, crustaceans, mollusks, aquatic plants, and algae. In some embodiments, the aquatic animals may include an aquatic species that is present, either fully or partially, in an aquatic environment, such as one or more of aquaculture fish and invertebrates.

In embodiments, the feed compositions may be used as a standalone feed or as additives and thus the term feed compositions includes feed additive compositions. In some embodiments, the feed compositions are used as feed additives for aquaculture environments. For example, aquaculture environments may include, but are not limited to, any type of water environment, including seawater, saltwater, freshwater, running water, brackish, and any combination thereof. For example, aquaculture systems may include, but are not limited to, one or more of raceways, tanks, and ponds. In an embodiment, the feed compositions may be administered as a topical application such as a paste either to feed or to treat aquatic animals, among other things.

Vibrio is a genus of Gram-negative bacteria, possessing a curved-rod shape (comma shape), several species of which can cause foodborne infection, usually associated with eating undercooked seafood. Several species of Vibrio are human pathogens. Most disease-causing strains are associated with gastroenteritis, but can also infect open wounds and cause septicemia. They can be carried by numerous marine animals, such as crabs or prawns, and have been known to cause fatal infections in humans during exposure. Pathogenic Vibrio species include V. cholerae (the causative agent of cholera), V. parahaemolyticus, V. vulnificus, V. alginolyticus, V. fluvialis, V. mimicus, V. harveyi, V. metschnikovii, V. furnissii, V. metoecus, and V. navarrensis (CDC, National Enteric Disease Surveillance, COVIS Annual Summary, 2014). V. cholerae is generally transmitted by contaminated water.

Pathogenic Vibrio species can cause foodborne illness (infection), usually associated with eating undercooked seafood. The pathogenic features can be linked to quorum sensing where bacteria are able to express their virulence factors via their signaling molecules.

Foodborne Vibrio infections are most often associated with eating raw shellfish. V. vulnificus can cause gastroenteritis when ingested in oysters, septicemia in undercooked shellfish, and necrotic wounds are found with 25% mortality, 50% with septicemia.

Vibrio species cause diseases in diverse marine animals reared in aquaculture. Vibrio harveyi causes luminescent vibriosis in shrimp and lobsters; V. vulnificus, V. anguillarum, and V. alginolyticus infect several fish species; and V. crassostreae infects oysters. In addition, Vibrio species (V. coralliilyticus and V. shiloi) cause coral bleaching and tissue necrosis in coral species in reefs world-wide.

“Effective amount” refers to an amount of an inventive compound, drug, or pharmaceutical agent that elicits the biological or medicinal response in an aquatic animal or a subject (i.e. a tissue system, animal or human) that is being sought by a researcher, veterinarian, medical doctor or other clinician, which includes, but is not limited to, alleviation of the symptoms of the disease or disorder being treated. In one aspect, the effective amount is that amount of an active which may treat or alleviate the disease or symptoms of the disease at a reasonable benefit/risk ratio applicable to any medical treatment. In another aspect, the effective amount is that amount of an inactive prodrug which when converted through normal metabolic processes to produce an amount of active drug capable of eliciting the biological or medicinal response in an aquatic animal or subject that is being sought.

It is also appreciated that the dose, whether referring to monotherapy or combination therapy, is advantageously selected with reference to any toxicity, or other undesirable side effects, that might occur during administration of one or more of the antimicrobial peptides described herein. Further, it is appreciated that the co-therapies described herein may allow for the administration of lower doses of antimicrobial peptides that show such toxicity, or other undesirable side effects, where those lower doses are below thresholds of toxicity or lower in the therapeutic window than would otherwise be administered in the absence of a co-therapy.

“Administering” includes all means of introducing the compounds and compositions described herein to the subject, including, but are not limited to, oral (po), intravenous (iv), intramuscular (im), subcutaneous (sc), transdermal, inhalation, buccal, ocular, sublingual, vaginal, rectal, and the like. The compounds and compositions described herein may be administered in unit dosage forms and/or formulations containing conventional nontoxic pharmaceutically-acceptable carriers, adjuvants, and/or vehicles.

Suitable dosages of the compounds can be determined by standard methods, for example by establishing dose-response curves in laboratory animal models or in humans in clinical trials. Illustratively, suitable dosages of compounds (administered in a single bolus or over time) include from about 1 pg/kg to about 10 g/kg, from about 1 pg/kg to about 1 μg/kg, from about 100 pg/kg to about 500 ng/kg, from about 1 pg/kg to about 1 ng/kg, from about 1 pg/kg to about 500 pg/kg, from about 100 pg/kg to about 500 ng/kg, from about 100 pg/kg to about 100 ng/kg, from about 1 ng/kg to about 10 mg/kg, from about 1 ng/kg to 1 mg/kg, from about 1 ng/kg to about 1 g/kg, from about 1 ng/kg to about 500 ng/kg, from about 100 ng/kg to about 500 μg/kg, from about 100 ng/kg to about 100 μg/kg, from about 1 μg/kg to about 500 μg/kg, or from about 1 μg/kg to about 100 μg/kg. In each of these embodiments, dose/kg refers to the dose per kilogram of a subject's or animal's mass or body weight.

Fish feed formulations may comprise from about 1 to about 2500 mg of an inventive compound or a physiologically acceptable derivative or salt thereof in association with and per kg of a fish feed composition.

In aquaculture, a practical mode of delivering a substance is in the feed. Indeed, fish feeds are a standard article of commerce, often tailored for an individual species. Typically, the feed is in the form of powder, particles, crumbles and pellets depending on the particular fish species, stage of development and other factors known to those skilled in the art. Therefore, in practicing the present invention, while other routes of delivery can be employed, the preferred method of delivery is in or on a fish feed and preferably a nutritionally balanced fish feed. The compound or physiologically acceptable derivative or salt is dispersed in or top-dressed onto the fish feed by known techniques.

“Feed” is generally used to describe a product which meets the daily nutritional needs of the fish being fed with it, that is, it contains all the essential nutrients. The term “feedstuff” in comparison is used to refer to a component of the complete feed, such as protein or fish oil or a component containing the necessary proteins and oils but without the proper vitamin or mineral content. The term nutritionally balanced or complete includes both complete feeds and feedstuffs. Fish feeds are generally manufactured to a formula specific for the aquatic target species being fed and intended aquatic production system.

While most temperate freshwater diets may be largely based upon the use of plant protein and energy sources, and cold water marine diets are largely based upon the use of fishmeal and other fishery by-products, there can be regional differences that reflect optimal use of locally available and/or least-cost formulation of ingredients.

Generally, substances that may be included in fish feed and feedstuffs include fish meal, fish silage (hydrolyzed fish), plant carbohydrate (such as wheat meal, corn meal, soy meal, etc.), fish oil, plant oil, coloring agents, vitamins, minerals, pharmaceuticals (such as antibiotics, growth promoters, etc.), and plant proteins, especially storage proteins including gluten.

These additional substances may serve to provide a balanced diet for the fish fed with the nutritional composition; they may serve to adjust the lipid/protein balance, fish or plant oils may be used to increase lipid content; they may, like the coloring agents, be used to make the flesh of farmed fish more closely resemble that of wild fish, which is particularly desirable for farmed salmon; or they may serve to improve or protect the health of the creature receiving the feed. However, the use of plant storage proteins, in particular gluten is desirable as it improves the texture, physical strength and lipid retention ability of the product.

Thus with such additional substances included, the product is a complete feed, especially a feed in pellet form or a feed or feedstuff in granular form (such as in powder, grain or meal form) comprising from about 1 to about 2500 mg of compound or a physiologically acceptable derivative or salt thereof per kg of feed or feedstuff.

Typically, the protein content will be 30 to 60% by weight, preferably 35 to 58%, more preferably 40 to 55% on a dry weight. The product will preferably have a lipid content of 8 to 35% by weight on a dry weight basis, more preferably 10 to 30%.

Vitamins, coloring agents, pharmaceuticals and minerals will generally form only a minor portion of the product, such as up to 10% by weight on a dry solids basis. Appropriate amounts can readily be calculated from the appropriate dosages and feed consumption rates for the fish receiving the feed.

Carbohydrates, such as digestible plant starch, for example wheat starch, will generally constitute up to 20% by weight on a dry weight basis of the product, preferably 5 to 15%. The water content of the feed will be 0.5 to 10% for a dry feed, preferably 2 to 9% and more particularly 3 to 8%. For a wet feed, the water content will be greater than 10% to 70%.

The invention further contemplates a solid feed comprising a formulation of the present invention containing an inventive compound and fish feed which may be administered to fish or diluted with fish feed matter to afford a complete feed composition.

The amount of compound or physiologically acceptable derivative or salt to be employed will vary with the specific improvement desired, the fish species, the age of the fish, and other factors known to those in the field of aquaculture. In general, a concentration in or on the feed of from about 1 to about 2500 mg per kg of fish feed will provide good results. In many instances, concentrations in the range of about 75 to about 2250 mg per kg will suffice.

For treatment purposes, pharmaceutical compositions comprising the compounds described herein may further comprise one or more pharmaceutically-acceptable excipients. A pharmaceutically-acceptable excipient is a substance that is non-toxic and otherwise biologically suitable for administration to a subject. Such excipients facilitate administration of the compounds described herein and are compatible with the active ingredient. Examples of pharmaceutically-acceptable excipients include stabilizers, lubricants, surfactants, diluents, anti-oxidants, binders, coloring agents, bulking agents, emulsifiers, or taste-modifying agents. In preferred embodiments, pharmaceutical compositions according to the invention are sterile compositions. Pharmaceutical compositions may be prepared using compounding techniques known or that become available to those skilled in the art.

Sterile compositions are also contemplated by the invention, including compositions that are in accord with national and local regulations governing such compositions.

The pharmaceutical compositions and compounds described herein may be formulated as solutions, emulsions, suspensions, or dispersions in suitable pharmaceutical solvents or carriers, or as pills, tablets, lozenges, suppositories, sachets, dragees, granules, powders, powders for reconstitution, or capsules along with solid carriers according to conventional methods known in the art for preparation of various dosage forms. Pharmaceutical compositions of the invention may be administered by a suitable route of delivery, such as oral, parenteral, rectal, nasal, topical, or ocular routes, or by inhalation. Preferably, the compositions are formulated for intravenous or oral administration.

For oral administration, the compounds the invention may be provided in a solid form, such as a tablet or capsule, or as a solution, emulsion, or suspension. To prepare the oral compositions, the compounds of the invention may be formulated to yield a dosage of, e.g., from about 0.1 mg to 1 g daily, or about 1 mg to 50 mg daily, or about 50 to 250 mg daily, or about 250 mg to 1 g daily. Oral tablets may include the active ingredient(s) mixed with compatible pharmaceutically acceptable excipients such as diluents, disintegrating agents, binding agents, lubricating agents, sweetening agents, flavoring agents, coloring agents and preservative agents. Suitable inert fillers include sodium and calcium carbonate, sodium and calcium phosphate, lactose, starch, sugar, glucose, methyl cellulose, magnesium stearate, mannitol, sorbitol, and the like. Exemplary liquid oral excipients include ethanol, glycerol, water, and the like. Starch, polyvinyl-pyrrolidone (PVP), sodium starch glycolate, microcrystalline cellulose, and alginic acid are exemplary disintegrating agents. Binding agents may include starch and gelatin. The lubricating agent, if present, may be magnesium stearate, stearic acid, or talc. If desired, the tablets may be coated with a material such as glyceryl monostearate or glyceryl distearate to delay absorption in the gastrointestinal tract and/or the tablets may be coated with an enteric coating.

Capsules for oral administration include hard and soft gelatin capsules. To prepare hard gelatin capsules, active ingredient(s) may be mixed with a solid, semi-solid, or liquid diluent. Soft gelatin capsules may be prepared by mixing the active ingredient with water, an oil, such as peanut oil or olive oil, liquid paraffin, a mixture of mono and di-glycerides of short chain fatty acids, polyethylene glycol 400, or propylene glycol.

Liquids for oral administration may be in the form of suspensions, solutions, emulsions, or syrups, or may be lyophilized or presented as a dry product for reconstitution with water or other suitable vehicle before use. Such liquid compositions may optionally contain: pharmaceutically-acceptable excipients such as suspending agents (for example, sorbitol, methyl cellulose, sodium alginate, gelatin, hydroxyethylcellulose, carboxymethylcellulose, aluminum stearate gel and the like); non-aqueous vehicles, e.g., oil (for example, almond oil or fractionated coconut oil), propylene glycol, ethyl alcohol, or water; preservatives (for example, methyl or propyl p-hydroxybenzoate or sorbic acid); wetting agents such as lecithin; and, if desired, flavoring or coloring agents.

For parenteral use, including intravenous, intramuscular, intraperitoneal, intranasal, or subcutaneous routes, the agents of the invention may be provided in sterile aqueous solutions or suspensions, buffered to an appropriate pH and isotonicity or in parenterally acceptable oil. Suitable aqueous vehicles include Ringer's solution and isotonic sodium chloride. Such forms may be presented in unit-dose form such as ampoules or disposable injection devices, in multi-dose forms such as vials from which the appropriate dose may be withdrawn, or in a solid form or pre-concentrate that can be used to prepare an injectable formulation. Illustrative infusion doses range from about 1 to 1000 μg/kg/minute of agent admixed with a pharmaceutical carrier over a period ranging from several minutes to several days.

For nasal, inhaled, or oral administration, the inventive pharmaceutical compositions may be administered using, for example, a spray formulation also containing a suitable carrier. The inventive compositions may be formulated for rectal administration as a suppository.

For topical applications, the compounds of the present invention are preferably formulated as creams or ointments or a similar vehicle suitable for topical administration. For topical administration, the inventive compounds may be mixed with a pharmaceutical carrier at a concentration of about 0.1% to about 10% of drug to vehicle. Another mode of administering the agents of the invention may utilize a patch formulation to effect transdermal delivery.

As used herein, the terms “treat” or “treatment” encompass both “preventative” and “curative” treatment. “Preventative” treatment is meant to indicate a postponement of development of a disease, a symptom of a disease, or medical condition, suppressing symptoms that may appear, or reducing the risk of developing or recurrence of a disease or symptom. “Curative” treatment includes reducing the severity of or suppressing the worsening of an existing disease, symptom, or condition. Thus, treatment includes ameliorating or preventing the worsening of existing disease symptoms, preventing additional symptoms from occurring, ameliorating or preventing the underlying systemic causes of symptoms, inhibiting the disorder or disease, e.g., arresting the development of the disorder or disease, relieving the disorder or disease, causing regression of the disorder or disease, relieving a condition caused by the disease or disorder, or stopping the symptoms of the disease or disorder.

The compounds of the present invention may be prepared by a variety of procedures known to one of ordinary skill in the art, some of which are illustrated in the schemes, preparations, and examples below. One of ordinary skill in the art recognizes that the specific synthetic steps for each of the routes described may be combined in different ways, or in conjunction with steps from different schemes, to prepare compounds of the invention. The products of each step in the schemes below can be recovered by conventional methods well known in the art, including extraction, evaporation, precipitation, chromatography, filtration, trituration, and crystallization. In the schemes below, all substituents unless otherwise indicated, are as previously defined. The reagents and starting materials are readily available to one of ordinary skill in the art. Others may be made by standard techniques of organic and heterocyclic chemistry which are analogous to the syntheses of known structurally-similar compounds and the procedures described herein which follow including any novel procedures.

Vibrio species are principal pathogens of marine animals, including fish, shellfish, and coral. Global warming and the concomitant rise of ocean temperatures correlates with increases in Vibrio prevalence and spread to regions beyond their typical equatorial habitats (Le Roux et al., 2015, Newton et al., 2012, Vezzulli et al., 2016, Coly et al., 2013, Martinez-Urtaza et al., 2013), consequently harming fish and shellfish aquaculture industries and natural marine ecosystems worldwide. Thus, there is a global need for new treatments for vibriosis in coral reef ecosystems, aquaculture, and in human health, due to consumption of contaminated fish and shellfish. In pathogenic marine Vibrio species studied to date, the bacterial cell-cell signaling system called quorum sensing controls biofilm formation, as well as expression and secretion of virulence factors (reviewed in Girard, 2019, Rutherford & Bassler, 2012, Ng & Bassler, 2009). Quorum sensing involves the production and detection of signaling molecules called autoinducers that provide information about the number and type of bacterial cells in the near vicinity. As populations of cells grow denser, autoinducer concentrations increase, and detection of these molecules drives changes in gene expression to alter population-wide behaviors, including those required for pathogenesis.

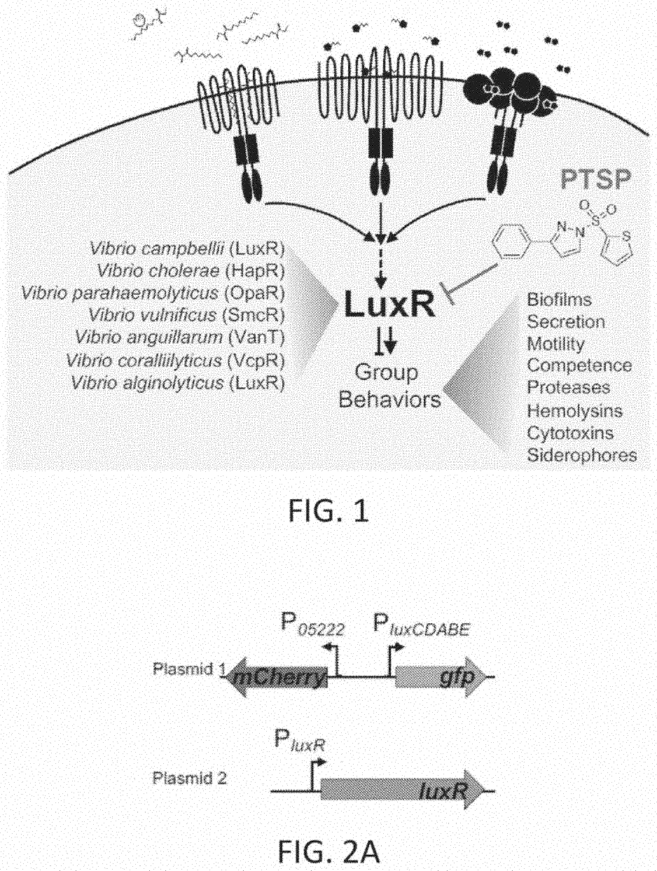

In vibrios, autoinducers are sensed by membrane-bound histidine kinase receptors that participate in a phosphorylation cascade, ultimately controlling production of the master regulator LuxR (FIG. 1) (reviewed in Ball & van Kessel, 2019, Ng & Bassler, 2009, Rutherford & Bassler, 2012). At low cell densities (LCD), the receptors do not bind autoinducers and function as kinases, shuttling phosphate through LuxU to the Sigma-54-dependent LuxO response regulator. Phosphorylated LuxO activates transcription of the quorum regulatory small RNAs, the Qrrs, which together with the Hfq chaperone repress LuxR translation and activate AphA translation. Thus, at LCD, low levels of LuxR are produced and high levels of AphA are produced, leading to expression of genes for individual behaviors. At high cell densities (HCD), high concentrations of autoinducers results in binding of autoinducers to the receptors, which switches the activity of the receptors to phosphatases. Because LuxO is not phosphorylated, the Qrrs are not transcribed, and thus AphA is not produced and maximal LuxR protein is produced. The general structure of the V. campbellii quorum sensing pathway depicted in FIG. 1 is conserved in numerous Vibrio species, although the autoinducer synthases/receptors and the number of Qrrs vary. For example, the luxMN genes are primarily present only in the Harveyi clade (Simpson et al., 2021), but the vast majority of Vibrio species encode LuxO (Girard, 2019). In addition, a nitric oxide sensor pair H—NOX/HqsK that also feeds phosphate into the system has been identified in some Vibrio species.

In each Vibrio species in which quorum sensing has been studied, the signaling cascade culminates in the master LuxR-type transcription factor, which activates and represses many genes ranging from ˜100 to ˜400 genes depending on the species (FIG. 1) (reviewed in Ball et al., 2017, Girard, 2019). The LuxR protein is highly conserved in all pathogenic vibrios studied to date (Rutherford et al., 2011, Kernell Burke et al., 2015, Kim et al., 2013, Lee et al., 2008, Tsou et al., 2009, Ball et al., 2017, Kim et al., 2010, De Silva et al., 2007). Although the naming of the V. campbellii protein LuxR causes confusion, this protein does not resemble or function like the LuxR protein that is part of the Vibrio fischeri LuxI/LuxR quorum sensing system, which requires binding to the autoinducer molecule made by LuxI for activity (Fuqua et al., 1994). Conversely, the LuxR from V. campbellii belongs to the TetR superfamily (Ramos et al., 2005), and these proteins are structurally, genetically, biochemically, and functionally distinct from the V. fischeri LuxR. LuxR/TetR homologs in vibrios include SmcR in V. vulnificus, HapR in V. cholerae, OpaR in V. parahaemolyticus, and VcpR in Vibrio coralliilyticus, which share 76-96% amino acid identity (Ball et al., 2017). LuxR/TetR proteins in vibrios directly bind to multiple sites in promoter regions and interact with other proteins (e.g., RNA polymerase, IHF) or compete with other proteins (e.g., H—NS) to activate or repress transcription of hundreds of quorum sensing genes. It is critical to note that these quorum-sensing LuxR/TetR-type proteins do not have a known ligand, although a putative ligand binding pocket has been defined in structures and shown to bind inhibitors. Thus, if there is a native ligand, it is also unknown how it affects function of the LuxR protein and its impact on the physiology and group behaviors of the cells.

Vibrio LuxR/TetR-type proteins play crucial roles in colonization and infection of hosts through quorum-directed regulation of biofilm formation, type III and type VI secretion systems, motility, and production of proteases, hemolysins, siderophores, and cytotoxin. Deletion or inhibition of LuxR proteins in several vibrios reduces or eliminates colonization and toxicity, effectively increasing host survival. Thus, LuxR represents a key target for designing therapeutics to block quorum sensing in vibrios. LuxR inhibitors would presumably render Vibrio cells unresponsive to quorum sensing signals even at HCD, thus restricting cells to their LCD gene expression program. Indeed, recent studies have shown that quorum sensing inhibitors are viable alternative approaches to traditional antibiotics in disease treatment with demonstrated efficacy in animal models. Because quorum sensing inhibitors typically do not affect growth of the bacteria but rather inhibit specific pathways these molecules are hypothesized to generate less selective pressure for evolving resistance.

V. campbellii LuxR-specific inhibitors have been identified via a variety of methods, including both in vitro and in silico screening strategies. These include compounds with varying functional groups such as aromatic enones, sulfonamides, sulfones, cinnamaldehydes, furanones, and brominated thiophenones and each was shown to specifically inhibit bioluminescence, biofilm formation, and/or protease activity in V. campbellii and in some cases other Vibrio species. However, either the maximum inhibition of bioluminescence for some molecules was low (˜3-fold) or the inhibitory concentrations required to observe phenotypic effects in vitro or in vivo were high (>20 mM). Several of these molecules have shown favorable therapeutic potential because addition of these molecules to brine shrimp larvae infected with Vibrio cells increased survival. In particular, the molecule called Qstatin (1-((5-bromothiophen-2-yl)sulfonyl)-1H-pyrazole) is a highly promising molecule that was shown to be a specific inhibitor of the LuxR homolog SmcR in Vibrio vulnificus both in vitro and in vivo (Kim et al., 2018).

Here, we used an E. coli bioassay to screen chemical libraries to identify specific inhibitors of LuxR that do not affect bacterial cell growth. Our structure-activity relationship data show that multiple thoiphenesulfonamide-containing molecules with heterocycle variations are strong inhibitors of LuxR-type proteins in a wide-range of pathogenic Vibrio species. Our data also show that LuxR homologs are more functionally distinct from each other than previously hypothesized.

### Results

### A Dual-Color Bioassay Screen Identifies LuxR Chemical Inhibitors

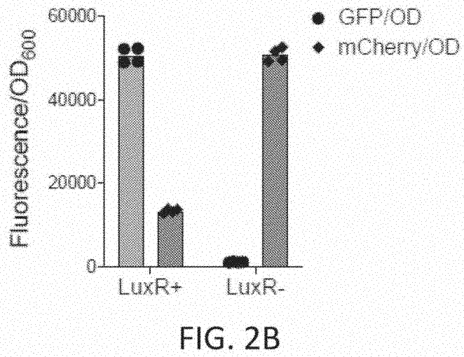

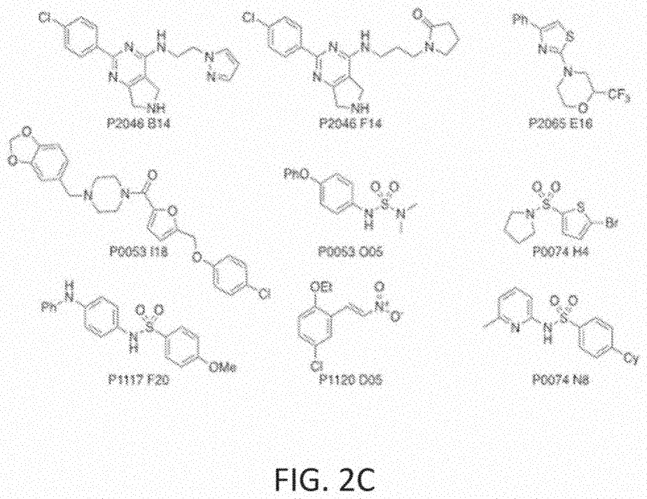

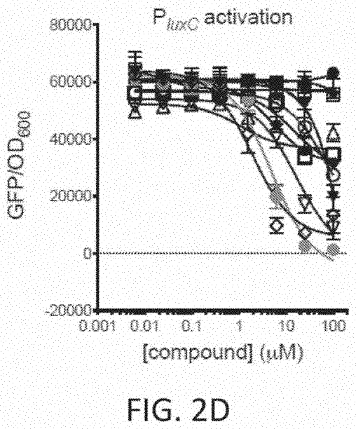

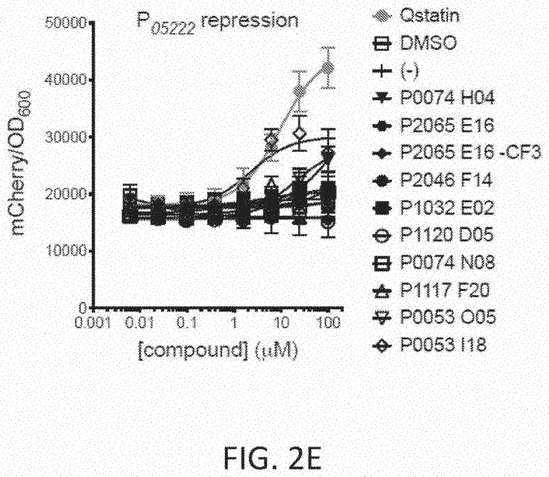

LuxR proteins have been shown to activate and repress gene expression in vibrios through direct binding to specific DNA sequences in promoters. Previously, we developed a bioassay that consists of a dual-color fluorescent reporter plasmid that reports both LuxR activities: the luxCDABE promoter is activated by LuxR and drives expression of gfp, and the VIBHAR_05222 promoter is repressed by LuxR and drives expression of mCherry (van Kessel et al., 2013b) (FIG. 2A). We used this reporter plasmid in an E. coli strain that also contains a plasmid expressing LuxR from its native promoter. Thus, in the presence of LuxR, GFP levels increased and mCherry levels decreased compared to the control strain (FIG. 2B). As a positive control, we showed that Qstatin inhibited LuxR activity in this assay. We screened ˜60,000 molecules in the ChemBridge and Chemdiv libraries and identified nine compounds that inhibited LuxR activation and/or repression but did not affect the final growth yield more than 10%. Four of these compounds contained a sulfonamide or sulfamide core with variable groups on each side similar to Qstatin, whereas the other compounds were structurally dissimilar (FIG. 2C). We synthesized or purchased these molecules and determined the half-maximal inhibitory concentration (IC₅₀) for each in the E. coli bioassay strain using titration curves (Table 2, FIG. 2D, 2E). P0053I18 had the best inhibitory effect on LuxR in the E. coli bioassay with an IC₅₀ similar to Qstatin (Table 2, FIG. 2D, 2E). P2065 E16 had only a minor inhibition of LuxR, and a variation of this molecule lacking the CF₃ group on the heterocycle had no activity (FIG. 2D, 2E). We also observed consistent inhibition by P0074 H04 and P0053 O05, which also contain sulfamide/sulfonamide cores, though the IC₅₀ values for these were higher than Qstatin or P0053 I18 (FIG. 2D, 2E).

### Structure-Activity Relationship Study of Thiophenesulfonamide Panel

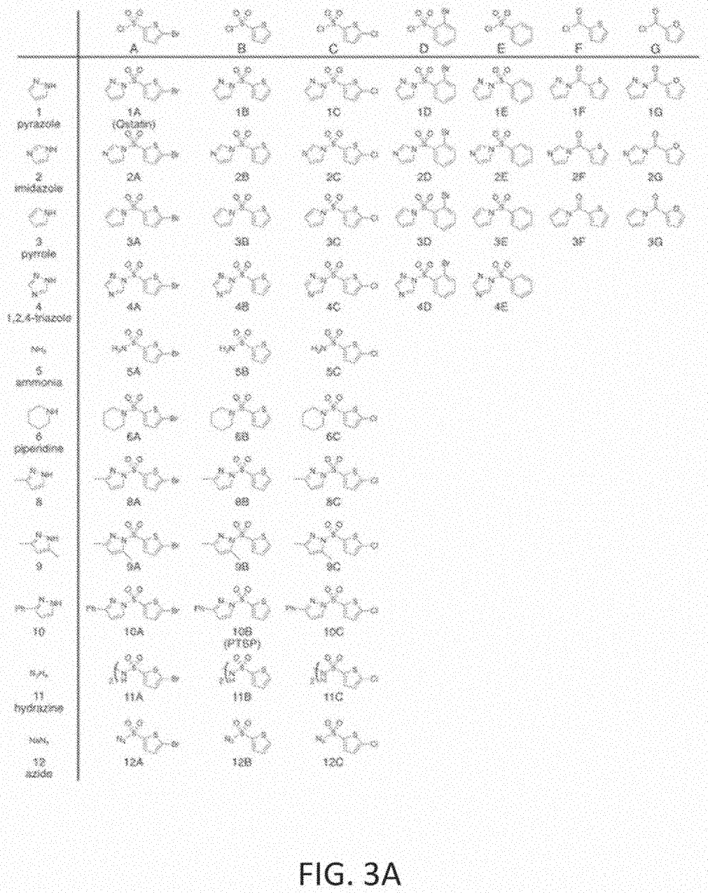

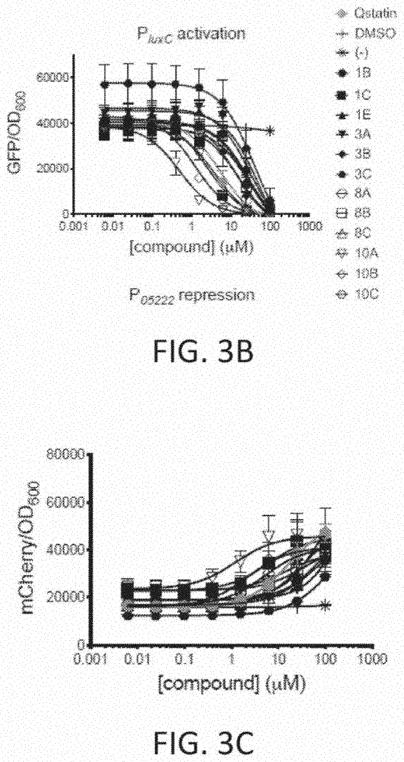

To further explore the thiophenesulfonamide class of compounds that includes Qstatin, P0053 O05, and P0074 H4, we performed a structure-activity relationship (SAR) study of Qstatin derivatives. We chose to focus on these thiophenesulfonamide molecules for SAR instead of P0053 I18 because the synthesis of sulfonamides is a single step, and the heterocycle components are readily available commercially. We synthesized a panel of Qstatin derivatives with steric and electronic structural variations in both heterocycles (FIG. 3A). Modifications to the heteroaromatic amine ring (pyrazole in the case of Qstatin) are referred to with number designations for each class and modifications to the thiophene ring with letter designations for each class (FIG. 3A). Assays with these molecules in the E. coli bioassay showed that the most active compounds contained a 3-methyl- (class 8) or 3-phenyl-substituted pyrazole (class 10), an unsubstituted pyrazole (class 1), or a pyrrole in place of the pyrazole (class 3) (FIG. 3B, 3C S2, S3). Compounds containing other heterocycles did not have activity against LuxR, It is particularly noteworthy that the compounds containing an imidazole ring (class 2) were not active, which was not expected given that the structure is highly similar to pyrroles and pyrazoles (classes 1 and 3). Methyl substitution at the 3 position of the pyrazole did not alter activity compared to Qstatin (class 8), however methyl substitutions at both the 3- and 5-positions of the pyrazole eliminated activity (class 9). In most cases, the presence/absence of Br or Cl atoms on the thiophene ring did not alter activity (FIG. 3A). For example, Qstatin, 1B, and 1C had similar activities, and 10A, 10B, and 10C had similar activities. The conformation of the sulfonamide core appeared to be critical because substitution with a carbonyl eliminated activity of that class of compounds (classes F and G). The most potent molecules were compounds 10A, 10B, and 10C, all of which contain a phenyl group on the 3-position of the pyrazole and vary in the presence or absence of bromine or chlorine on the thiophene ring (FIG. 3A, S2, S3).

2-thiophenesulfonamide Compounds Specifically Inhibit LuxR Proteins

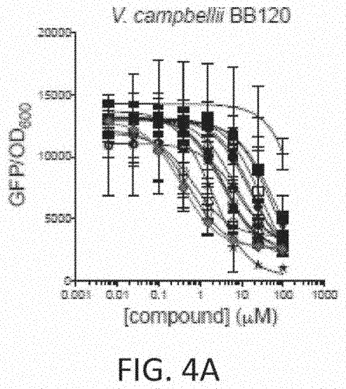

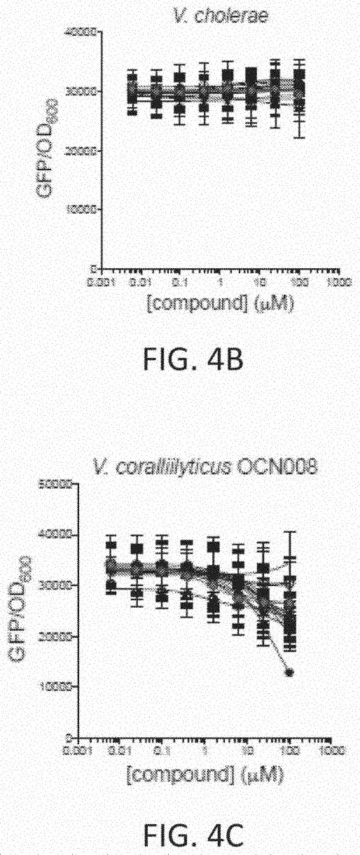

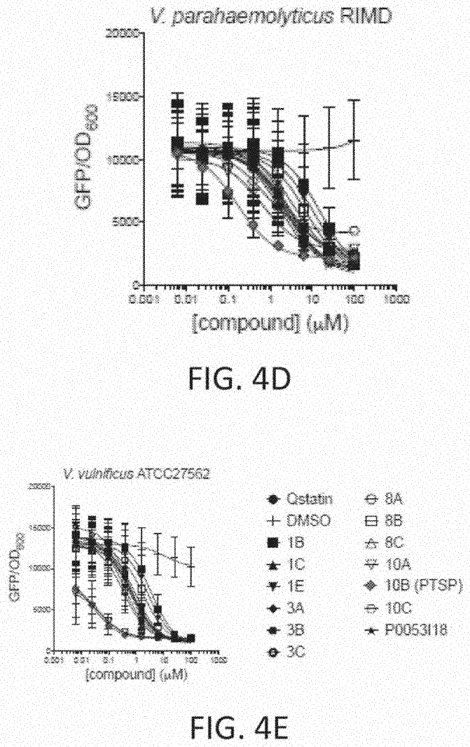

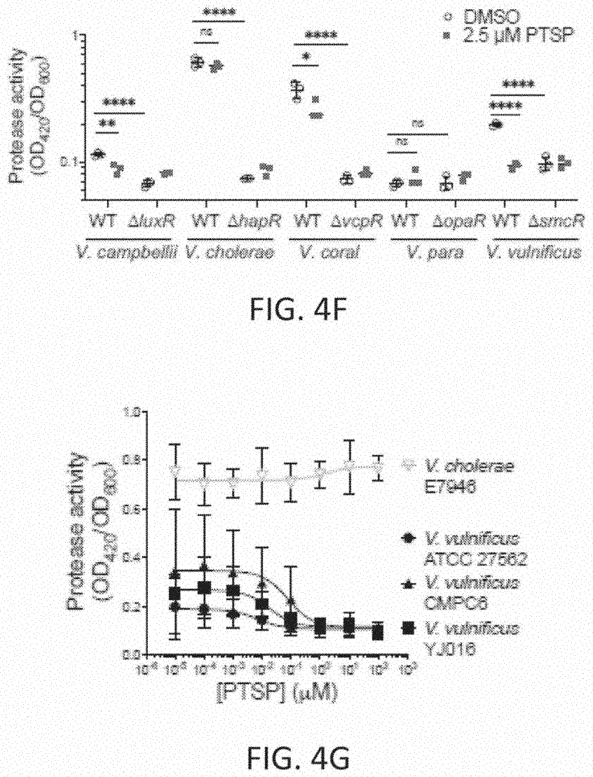

Many sulfonamide-containing compounds are known to target the folate synthesis pathway in bacteria and inhibit growth (bacteriostatic), and thus these “sulfa drugs” have been used as broad-spectrum antibiotics for decades (Hammoudeh et al., 2013, Roland et al., 1979). However, the structurally distinct 2-thiophenesulfonamides generally did not have any bacteriostatic activity; only two compounds (4A and 4C) in this entire panel limited E. coli growth yield more than 10% compared to negative controls, and this effect was only observed at concentrations of 100 mM and higher. Addition of high concentrations of 10B or 10C to V. campbellii or V. vulnificus did not alter growth rate or growth yield (FIG. 4C), suggesting that the inhibitory activity is specific to LuxR, and these are not general antibacterial compounds. We also tested the stability of the inhibitors in bacterial cultures and the timing of the inhibitory effect. We observed that molecule 10B inhibited LuxR activity rapidly; GFP production was blocked 120 minutes after 10B addition to the culture, and this was maintained through the longest timepoint tested at 16 hours.

RNA-seq performed with Qstatin added to V. vulnificus cultures showed that Qstatin has a specific effect on the SmcR regulon (Kim et al., 2018). The Qstatin-treated regulon overlapped with the SmcR regulon by more than 50%, with similar gene expression fold-changes. There were no significant off-target effects in the presence of Qstatin in the ΔsmcR strain, indicating that Qstatin specifically targets only SmcR. Finally, addition of Qstatin to wild-type V. vulnificus increases smcR transcript levels 2.5-fold; however, the authors did not observe a correlating increase in SmcR protein levels (Kim et al., 2018). This is important to note because LuxR and HapR proteins are known to autorepress their own expression. To test the effects of adding inhibitors to V. campbellii LuxR, we added 10A, 10B, and 10C to wild-type V. campbellii and used qRT-PCR and western blots to examine the effects on luxR transcript and protein levels. We also examined the transcript levels for luxC and 05222 genes that are directly activated or repressed by LuxR, respectively. We observed a significant 6- to 7-fold increase in luxR transcript levels in the presence of each of the 10A-C inhibitors, as well as a visible increase in LuxR protein level). Addition of 10A-C to wild-type cultures significantly decreased luxC transcripts to levels in a ΔluxR strain. Similarly, addition of 10A-C to wild-type cultures significantly increased 05222 transcripts. Collectively, these data show that 2-thiophenesulfonamide molecules are stable in bacterial culture, specifically inhibit LuxR activity, and do not significantly affect cell growth.

2-thiophenesulfonamides Inhibit Quorum Sensing Gene Expression in Vibrios

Qstatin has been shown to be an effective inhibitor of SmcR in vitro and in vivo (Kim et al., 2018). It also inhibits pathogenesis in V. campbellii, V. parahaemolyticus, and V. vulnificus in a shrimp infection assay, presumably through inhibition of the LuxR-type protein in these strains (Kim et al., 2018). To assess the activity of our panel of thiophenesulfonamide inhibitors against other vibrios, we assayed quorum-sensing controlled phenotypes in several strains from five Vibrio species: V. campbellii, V. coralliilyticus, V. cholerae, V. parahaemolyticus, and V. vulnificus. Although most of these vibrios are not bioluminescent, the luxCDABE operon from V. campbellii BB120 is routinely used to measure quorum sensing and LuxR regulation in other vibrios because LuxR directly binds this promoter and is required for gene expression. We introduced a plasmid containing the luxCDABE promoter driving expression of gfp (pCS19 (kanamycin-resistance cassette) or pCS42 (gentamicin-resistance cassette)) into each of the Vibrio strains. We focused our assays on the molecules with the most activity determined in FIG. 3A. We observed that 10A, 10B, and 10C molecules were the most inhibitory in V. campbellii, V. vulnificus, and V. parahaemolyticus, and in each, 8A had a similar IC₅₀ to Qstatin (FIG. 4A-E, Table 3). Molecules 10A, 10B, and 10C are so potent in V. vulnificus that we performed extended serial dilutions to obtain accurate IC₅₀ data because initial titrations did not yield enough points for a complete curve (FIG. 4E, Table 3). Notably, 10A, 10B, and 10C were much more inhibitory compared to Qstatin, with IC₅₀ values that were 30- to >500 lower than Qstatin in V. vulnificus (Table 3). We also observed that few molecules were active in V. coralliilyticus OCN008, but 3B had the most noticeable effect (FIG. 4C). Importantly, there was a distinct difference in the IC₅₀ for each molecule when compared across the five species. V. vulnificus exhibited very low IC₅₀ values for all the top panel molecules (FIG. 4E), whereas V. cholerae was completely resistant to these molecules even at high concentrations (FIG. 4B). Using 10B as an example, the IC₅₀ values were orders of magnitude different comparing V. vulnificus (0.002 mM) to V. campbellii (0.35 mM) (FIG. 4A, 4E, Table 3). To further examine this observation, we assayed the top panel of molecules against additional strain types for each species (Table 3). We observed that the inhibitory effect of the molecules was similar for each strain within a species (Table 3). For example, the IC₅₀ values for 10B for all three V. vulnificus clinical isolates ranged from 2-30 nM. From these data, we conclude that 10B has the highest inhibitory activity in most Vibrio species, with variation in the IC₅₀ that is specific to the Vibrio species tested.

We next focused on the best inhibitor in the panel, 10B, which is called 3-phenyl-1-(thiophen-2-ylsulfonyl)-1H-pyrazole, and thus we refer to 10B as PTSP from here on. We assessed LuxR function in the presence of PTSP by assaying protease production, a key virulence activity in vibrios during pathogenesis and host cell lysis (FIG. 4F, 4G). LuxR proteins have been shown to activate several genes encoding proteases in Vibrio species, such as HapA in V. cholerae, VvpE in V. vulnificus, PrtA in V. parahaemolyticus, and VcpA/VcpB in V. coralliilyticus (previously called VtpA and VtpB when the strain was misidentified as Vibrio tubiashii). We analyzed protease activity of bacterial supernatants using azocasein as a substrate and compared wild-type strains to their isogenic D/uxR counterparts for one representative strain for each species. In each species except V. parahaemolyticus, the wild-type strain produced significantly more protease activity than the D/uxR strain (FIG. 4F). Addition of PTSP to the wild-type strain significantly reduced protease activity in V. campbellii, V. coralliilyticus, and V. vulnificus, but not in V. cholerae (FIG. 4F). V. parahaemolyticus exhibited extremely low protease activity in this assay, thus the effect of PTSP on V. parahaemolyticus cannot be ascertained in this experiment. We observed that the effect of PTSP on protease activity mirrors that of bioluminescence: PTSP had minimal activity against V. coralliilyticus and highest activity against V. vulnificus. (FIG. 4F). Although we assessed the effect of PTSP on protease activity in numerous strains for V. campbellii, V. coralliilyticus, and V. parahaemolyticus, the protease activity was so low in many of these isolates that an IC₅₀ could not be reliably calculated for these. However, for the three V. vulnificus strains, we calculated the IC₅₀ values for PTSP inhibition and observed a similar range of protease inhibition as we observed for bioluminescence: ATCC 27562=6.8 nM, CMPC6=78.1 nM, and YJ016=18.3 nM (FIG. 4G). From the bioluminescence and protease assay data, we conclude that PTSP is a potent inhibitor of V. vulnificus, V. parahaemolyticus, and V. campbellii, with moderate effects on V. coralliilyticus. Inhibitor PTSP and derivatives were not active against V. cholerae (FIG. 4B, 4F, 4G). Further, we conclude that the use of the E. coli bioassay reporter is a valid assay for monitoring endogenous LuxR activity in these Vibrio species.

2-thiophenesulfonamide Efficacy is Driven by Amino Acid Conservation in the Ligand Binding Pocket

We hypothesized that there were several possible reasons for the observed difference in PTSP inhibition in the various Vibrio species, including but not limited to: 1) differences in LuxR-inhibitor interaction(s), 2) differences in diffusion of the inhibitor across the membranes, or 3) stability of the inhibitor in the cell and/or cell culture. To examine PTSP activity in a common strain background, we cloned the luxR gene from each Vibrio species into a plasmid under control of an IPTG-inducible promoter and assayed the effect of PTSP against these proteins in the E. coli bioassay. We found that HapR was unresponsive to PTSP in E. coli, exhibiting a similar level of GFP expression to the negative control Conversely, SmcR, LuxR, OpaR, and VcpR were each inhibited by PTSP with a trend similar to that observed in their native Vibrio cells, in which the order of sensitivity to PTSP inhibition was SmcR>LuxR>OpaR>VcpR. We conclude that the differences in PTSP activity in Vibrio species is due to differences in interaction between PTSP and the LuxR-type protein in each Vibrio.

Using the SmcR-Qstatin X-ray crystal structure as a guide (Kim et al., 2018), alignment of the LuxR-type proteins from each Vibrio species showed that there are four residues that interact with Qstatin in SmcR that are variable in HapR and/or VcpR. We therefore sought to determine if any of the four residues that differ between HapR and LuxR/SmcR in the putative ligand binding pocket are sufficient to render SmcR insensitive to PTSP. First, we introduced substitutions in SmcR to mimic the amino acid sequence of HapR: F75Y, I96L, V1401, and C170F. We observed that the substitutions of F75Y and C170F in SmcR abolished PTSP inhibition, suggesting that F75 and C170 are both necessary for PTSP inhibition of SmcR transcription regulation in vivo. However, SmcR I96L and V1401 were not significantly different from wild-type SmcR. Next, we introduced single substitutions in HapR to mimic the amino acid present in SmcR: Y76F, L97I, I141V, and F171C. However, none of these substitutions alone were sufficient to make HapR sensitive to PTSP. Further, combination of Y76F and F171C did not result in PTSP inhibition. From these results, we conclude that SmcR F75 and C170 are critical residues necessary for PTSP inhibition of SmcR activity.

Modeling of 2-thiophenesulfonamide Inhibitors Predicts Efficacy In Vivo

Previous studies have used molecular docking simulations to predict effective inhibitors of LuxR family proteins. To examine the accuracy of using molecular docking to predict effective inhibitors of LuxR proteins, we used Autodock Vina software & Olson, 2010) to simulate binding of molecules to the structure of SmcR and calculate the best binding mode and predicted binding energy (kcal/mol). First, to validate this modelling approach, we used Autodock Vina to predict the binding position of Qstatin into the SmcR (apo) X-ray crystal structure and compared it to the solved structure of SmcR-Qstatin. Although there was a slight shift (0.6 to 1.4 Å), the position and orientation of Qstatin within the putative ligand binding pocket of SmcR predicted by Autodock Vina was consistent with the SmcR-Qstatin structure. We next used Autodock Vina to predict the binding position of PTSP in both SmcR and HapR. We observed that PTSP was modeled in the opposite orientation in SmcR compared to HapR. We suspected that the orientation of PTSP was likely driven at least partially by the rotational position of glutamine 137 in SmcR (Q138 in HapR) that clashes with the phenyl ring of PTSP. We also noted that SmcR Q137 had multiple rotamers among the four SmcR chains within the asymmetric unit, and Autodock Vina modelling indicated that PTSP was oriented differently in SmcR chain A compared to chain B. Thus, the binding orientation and interactions of PTSP with SmcR are likely influenced, at least in part, by the orientation of Q137. The predicted orientation of PTSP was different in chains A and B that have different Q137 rotamers and different predicted binding energies (−7.5 kcal/mol for chain A compared to −0.2 kcal/mol for chain B). In addition, the predicted binding energies for PTSP in HapR were clearly worse at +1.6 kcal/mol for chain A and +10.4 kcal/mol for chain B.

Because Autodock Vina accurately predicted the binding position of Qstatin in SmcR, we modelled binding of all the molecules in the sulfonamide panel into SmcR. For the molecules that were active in vivo, we plotted the predicted binding affinities for each molecule against the calculated IC₅₀ values from the in vivo assay in V. vulnificus. We were unable to plot the remaining molecules because an IC₅₀ value could not be calculated from any in vivo data because titrations of these molecules did not produce an inhibition curve. Molecules 10A, 10B (PTSP), and 10C clustered in a group with the similarly low predicted binding energies and lowest observed IC₅₀ values. Conversely, the molecules with the low-moderate inhibitory activity were predicted to have similar binding energies that were indistinguishable. From these data, we conclude that molecular docking simulations can accurately predict sulfonamide molecules with tight binding affinity and strong inhibitory activity against SmcR and LuxR.

### In Vitro Analysis of PTSP Binding Affinity and Effects on DNA Binding Activity

To test the validity of the modelling predictions for PTSP, we performed isothermal titration calorimetry (ITC) to determine the binding affinity of SmcR and LuxR for PTSP. We found that PTSP had a lower (tighter) dissociation constant (K_{d}) for SmcR (0.23 μM) compared to LuxR (0.51 μM). As a control, we tested the binding affinity of Qstatin for SmcR and observed a dissociation constant of 0.74 μM (Table 4), which is slightly higher than previously published ITC experiments with Qstatin and SmcR that reported a 0.47 μM dissociation constant (Kim et al., 2018). We noted that PTSP binding to both LuxR and SmcR had lower entropy and enthalpy compared to SmcR binding to Qstatin. Further, PTSP binding to SmcR had a lower binding energy than Qstatin (Table 3), although the modeling predictions were overall higher binding energies than experimentally observed: −23 kJ/mol for Qstatin and −30 kJ/mol for PTSP determined by modeling compared to −35 kJ/mol for Qstatin and −38 kJ/mol for PTSP determined by ITC. Our collective data show that PTSP had a lower IC₅₀ in V. vulnificus than in V. campbellii, and PTSP had a tighter binding affinity to V. vulnificus SmcR than V. campbellii LuxR. Thus, we conclude that the efficacy of PTSP inhibition in vivo in these two Vibrio species correlates to the binding affinity determined in vitro for these two proteins. Further, our data suggest that the increased efficacy of PTSP inhibitory activity against SmcR is due to lower (tighter) binding affinity of PTSP to SmcR compared to Qstatin.

We noted that Qstatin and PTSP are both predicted to bind in the putative ligand binding pocket of SmcR. It is currently unknown how Qstatin allosterically affects DNA binding through its interactions in the ligand binding domain. Kim et al. showed that Qstatin does not appreciably alter the DNA binding constant for SmcR, but rather affects the entropy and enthalpy with which it interacts with DNA (Kim et al., 2018). We therefore also assessed the effect of sulfonamide PTSP on the DNA binding activity of LuxR and SmcR in vitro. Addition of saturating concentrations of PTSP did not alter DNA binding to LuxR or SmcR. This result is similar to the finding that Qstatin has very little effect on SmcR DNA binding affinity. (Kim et al., 2018). Collectively, these data show that thiophenesulfonamides inhibit the function of LuxR proteins to different levels. In addition, these data suggest that the mechanism of inhibition by thiophenesulfonamides is likely similar in both SmcR and LuxR.

TABLE 1
The following represent some, non-limiting, illustrative embodiments of
compounds of the invention and Formula (I):

| Compound | Structure | Name |
|---|---|---|
| P2065 E16 | 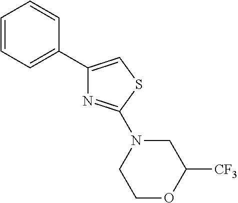 [SMILES: FC(F)(F)C1CN(c2nc(-c3ccccc3)cs2)CCO1] | 4-(4-phenylthiazol-2-yl)-2- (trifluoromethyl)morpholine |
| P0053 118 | 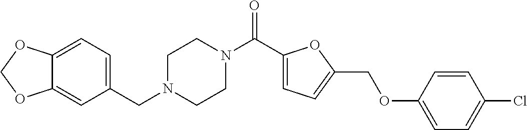 [SMILES: O=C(c1ccc(COc2ccc(Cl)cc2)o1)N1CCN(Cc2ccc3c(c2)OCO3)CC1] | (4-(benzo[d][1,3]dioxol-5- ylmethyl)piperazin-1-yl)(5-((4- chlorophenoxy)methyl)furan-2- yl)methanone |
| P0074 H04 | 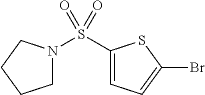 [SMILES: O=S(=O)(c1ccc(Br)s1)N1CCCC1] | 1-((5-bromothiophen-2- yl)sulfonyl)pyrrolidine |
| 1F | 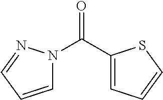 [SMILES: O=C(c1cccs1)n1cccn1] | (1H-pyrazol-1-yl)(thiophen-2- yl)methanone |
| 1G | 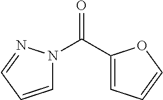 [SMILES: O=C(c1ccco1)n1cccn1] | furan-2-yl(1H-pyrazol-1- yl)methanone |
| 2A | 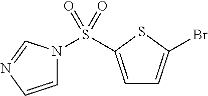 [SMILES: O=S(=O)(c1ccc(Br)s1)n1ccnc1] | 1-((5-bromothiophen-2- yl)sulfonyl)-1H-imidazole |
| 2B | 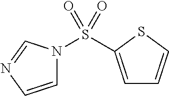 [SMILES: O=S(=O)(c1cccs1)n1ccnc1] | 1-(thiophen-2-ylsulfonyl)-1H- imidazole |
| 2C | 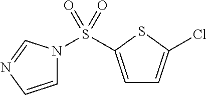 [SMILES: O=S(=O)(c1ccc(Cl)s1)n1ccnc1] | 1-((5-chlorothiophen-2- yl)sulfonyl)-1H-imidazole |
| 2E | 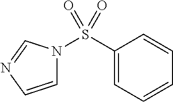 [SMILES: O=S(=O)(c1ccccc1)n1ccnc1] | 1-(phenylsulfonyl)-1H-imidazole |
| 2F | 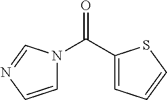 [SMILES: O=C(c1cccs1)n1ccnc1] | (1H-imidazol-1-yl)(thiophen-2- yl)methanone |
| 2G | 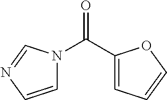 [SMILES: O=C(c1ccco1)n1ccnc1] | furan-2-yl(1H-imidazol-1- yl)methanone |
| 3D | 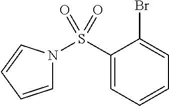 [SMILES: O=S(=O)(c1ccccc1Br)n1cccc1] | 1-((2-bromophenyl)sulfonyl)-1H- pyrrole |
| 3E | 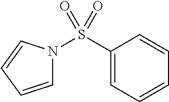 [SMILES: O=S(=O)(c1ccccc1)n1cccc1] | 1-(phenylsulfonyl)-1H-pyrrole |
| 3F | 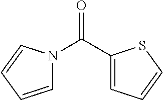 [SMILES: O=C(c1cccs1)n1cccc1] | (1H-pyrrol-1-yl)(thiophen-2- yl)methanone |
| 3G | 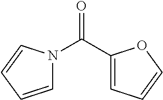 [SMILES: O=C(c1ccco1)n1cccc1] | furan-2-yl(1H-pyrrol-1- yl)methanone |
| 4B | 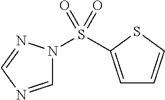 [SMILES: O=S(=O)(c1cccs1)n1cncn1] | 1-(thiophen-2-ylsulfonyl)-1H- 1,2,4-triazole |
| 5A | 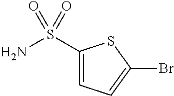 | 5-bromothiophene-2-sulfonamide |
| 5B | 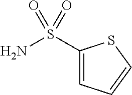 | thiophene-2-sulfonamide |
| 5C | 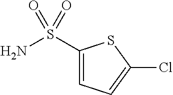 | 5-chlorothiophene-2-sulfonamide |
| 6A | 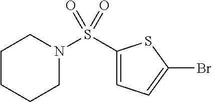 [SMILES: O=S(=O)(c1ccc(Br)s1)N1CCCCC1] | 1-((5-bromothiophen-2- yl)sulfonyl)piperidine |
| 6B | 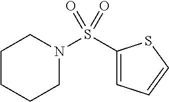 [SMILES: O=S(=O)(c1cccs1)N1CCCCC1] | 1-(thiophen-2- ylsulfonyl)piperidine |
| 6C | 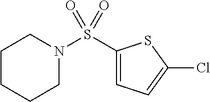 [SMILES: O=S(=O)(c1ccc(Cl)s1)N1CCCCC1] | 1-((5-chlorothiophen-2- yl)sulfonyl)piperidine |
| 7A | 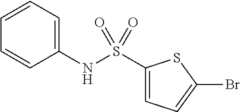 [SMILES: O=S(=O)(Nc1ccccc1)c1ccc(Br)s1] | 5-bromo-N-phenylthiophene-2- sulfonamide |
| 7B | 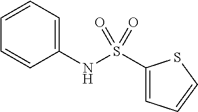 [SMILES: O=S(=O)(Nc1ccccc1)c1cccs1] | N-phenylthiophene-2- sulfonamide |
| 7C | 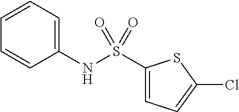 [SMILES: O=S(=O)(Nc1ccccc1)c1ccc(Cl)s1] | 5-chloro-N-phenylthiophene-2- sulfonamide |
| 9A | 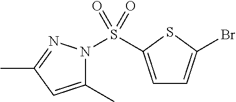 [SMILES: Cc1cc(C)n(S(=O)(=O)c2ccc(Br)s2)n1] | 1-((5-bromothiophen-2- yl)sulfonyl)-3,5-dimethyl-1H- pyrazole |
| 9B | 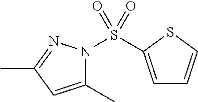 | 3,5-dimethyl-1-(thiophen-2- ylsulfonyl)-1H-pyrazole |
| 9C | 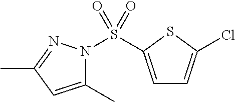 [SMILES: Cc1cc(C)n(S(=O)(=O)c2ccc(Cl)s2)n1] | 1-((5-chlorothiophen-2- yl)sulfonyl)-3,5-dimethyl-1H- pyrazole |
| 11A | 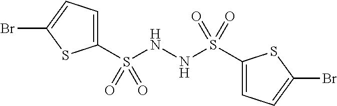 [SMILES: O=S(=O)(NNS(=O)(=O)c1ccc(Br)s1)c1ccc(Br)s1] | 5-bromo-N′-((5-bromothiophen-2- yl)sulfonyl)thiophene-2- sulfonohydrazide |
| 11B | 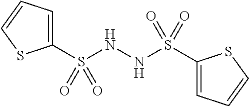 [SMILES: O=S(=O)(NNS(=O)(=O)c1cccs1)c1cccs1] | N′-(thiophen-2- ylsulfonyl)thiophene-2- sulfonohydrazide |
| 11C | 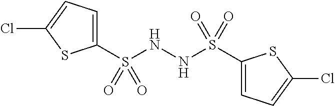 [SMILES: O=S(=O)(NNS(=O)(=O)c1ccc(Cl)s1)c1ccc(Cl)s1] | 5-chloro-N′-((5-chlorothiophen-2- yl)sulfonyl)thiophene-2- sulfonohydrazide |
| 12A | 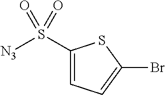 [SMILES: [N-]=[N+]=NS(=O)(=O)c1ccc(Br)s1] | 5-bromothiophene-2-sulfonyl azide |
| 12B | 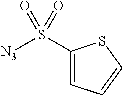 [SMILES: [N-]=[N+]=NS(=O)(=O)c1cccs1] | thiophene-2-sulfonyl azide |
| 12C | 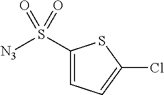 [SMILES: [N-]=[N+]=NS(=O)(=O)c1ccc(Cl)s1] | 5-chlorothiophene-2-sulfonyl azide |
| 1B | 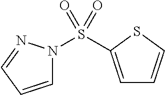 [SMILES: O=S(=O)(c1cccs1)n1cccn1] | 1-(thiophen-2-ylsulfonyl)-1H- pyrazole |
| 1C | 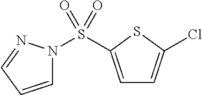 [SMILES: O=S(=O)(c1ccc(Cl)s1)n1cccn1] | 1-((5-chlorothiophen-2- yl)sulfonyl)-1H-pyrazole |
| 1D | 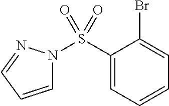 [SMILES: O=S(=O)(c1ccccc1Br)n1cccn1] | 1-((2-bromophenyl)sulfonyl)-1H- pyrazole |
| 1E | 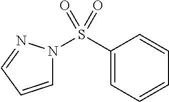 [SMILES: O=S(=O)(c1ccccc1)n1cccn1] | 1-(phenylsulfonyl)-1H-pyrazole |
| 1M | 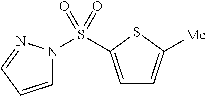 [SMILES: Cc1ccc(S(=O)(=O)n2cccn2)s1] | 1-((5-methylthiophen-2- yl)sulfonyl)-1H-pyrazole |
| 2D | 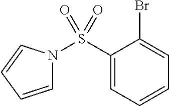 [SMILES: O=S(=O)(c1ccccc1Br)n1cccc1] | 1-((2-bromophenyl)sulfonyl)-1H- pyrrole |
| 3A | 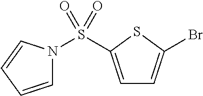 [SMILES: O=S(=O)(c1ccc(Br)s1)n1cccc1] | 1-((5-bromothiophen-2- yl)sulfonyl)-1H-pyrrole |
| 3B | 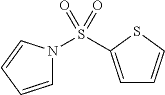 [SMILES: O=S(=O)(c1cccs1)n1cccc1] | 1-(thiophen-2-ylsulfonyl)-1H- pyrrole |
| 3C | 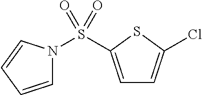 [SMILES: O=S(=O)(c1ccc(Cl)s1)n1cccc1] | 1-((5-chlorothiophen-2- yl)sulfonyl)-1H-pyrrole |
| 3M | 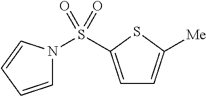 [SMILES: Cc1ccc(S(=O)(=O)n2cccc2)s1] | 1-((5-methylthiophen-2- yl)sulfonyl)-1H-pyrrole |
| 4A | 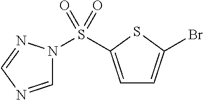 [SMILES: O=S(=O)(c1ccc(Br)s1)n1cncn1] | 1-((5-bromothiophen-2- yl)sulfonyl)-1H-1,2,4-triazole |
| 4C | 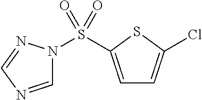 [SMILES: O=S(=O)(c1ccc(Cl)s1)n1cncn1] | 1-((5-chlorothiophen-2- yl)sulfonyl)-1H-1,2,4-triazole |
| 4D | 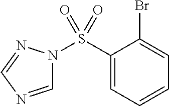 [SMILES: O=S(=O)(c1ccccc1Br)n1cncn1] | 1-((2-bromophenyl)sulfonyl)-1H- 1,2,4-triazole |
| 4E | 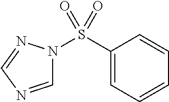 [SMILES: O=S(=O)(c1ccccc1)n1cncn1] | 1-(phenylsulfonyl)-1H-1,2,4- triazole |
| 4M | 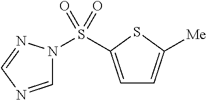 [SMILES: Cc1ccc(S(=O)(=O)n2cncn2)s1] | 1-((5-methylthiophen-2- yl)sulfonyl)-1H-1,2,4-triazole |
| 8A | 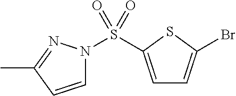 | 1-((5-bromothiophen-2- yl)sulfonyl)-3-methyl-1H-pyrazole |
| 8B | 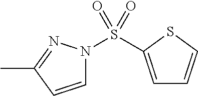 | 3-methyl-1-(thiophen-2- ylsulfonyl)-1H-pyrazole |
| 8C | 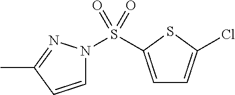 | 1-((5-chlorothiophen-2- yl)sulfonyl)-3-methyl-1H-pyrazole |
| 10A | 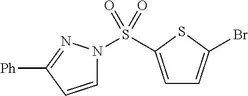 [SMILES: O=S(=O)(c1ccc(Br)s1)n1ccc(-c2ccccc2)n1] | 1-((5-bromothiophen-2- yl)sulfonyl)-3-phenyl-1H-pyrazole |
| 10B | 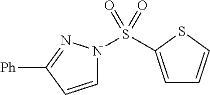 [SMILES: O=S(=O)(c1cccs1)n1ccc(-c2ccccc2)n1] | 3-phenyl-1-(thiophen-2- ylsulfonyl)-1H-pyrazole |
| 10C | 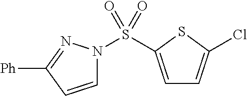 [SMILES: O=S(=O)(c1ccc(Cl)s1)n1ccc(-c2ccccc2)n1] | 1-((5-chlorothiophen-2- yl)sulfonyl)-3-phenyl-1H-pyrazole |
| 10M | 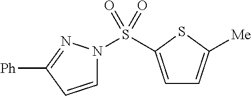 [SMILES: Cc1ccc(S(=O)(=O)n2ccc(-c3ccccc3)n2)s1] | 1-((5-methylthiophen-2- yl)sulfonyl)-3-phenyl-1H-pyrazole |

TABLE 2
Inhibition (IC₅₀) of GFP production by LuxR in E. coli bioassay.

| | | IC₅₀ 95% |
|---|---|---|
| | IC₅₀ | confidence |
| Molecule | (μM) | interval (μM) |
| P0053 I18 | 1.9 | 1.2-3.0 |
| P0053 O05 | 16.5 | 12.0-22.8 |
| P0074 H04 | ND | ND |
| P0074 N08 | 14.6 | 8.1-26.9 |
| P1032 E02 | ND | ND |
| P1117 F20 | 1.1 | 0.2-4.1 |
| P1120 D05 | 73.7 | 40.7-171.2 |
| P2046 F14 | ND | ND |
| P2065 E16 | 5.3 | 2.8-9.9 |
| Qstatin | 5.0 | 3.5-7.0 |

ND = a curve could not be fit to these data to determine IC₅₀.

TABLE 3
Inhibition (IC₅₀) of the P_{luxC-gfp} reporter in Vibrio strains.

| IC₅₀ | | | | | | | | | | | | | | |
|---|---|---|---|---|---|---|---|---|---|---|---|---|---|---|
| (μM)^{a} | Qstatin | 1B | 1C | 1E | 3A | 3B | 3C^{b} | 8A | 8B | 8C | 10A | 10B | 10C | P0053I18 |
| Vcamp | 4.0 | 52.6 | 3.3 | 51.4 | 15.0 | 18.9 | 8.3 | 3.8 | 32.4 | 6.7 | 0.58 | 0.35 | 0.71 | 2.0 |
| BB120 | | | | | | | | | | | | | | |
| Vcamp | 1.2 | 23.2 | 1.0 | 14.8 | 1.6 | 3.6 | ND | 1.2 | 8.7 | 2.7 | 0.40 | 0.10 | 0.16 | 4.3 |
| HY01 | | | | | | | | | | | | | | |
| Vcamp | 0.8 | 12.8 | 0.5 | 8.4 | 1.1 | 2.0 | ND | 0.7 | 3.9 | 1.5 | 0.05 | 0.03 | 0.09 | 1.8 |
| ATCC | | | | | | | | | | | | | | |
| 25920 | | | | | | | | | | | | | | |
| Vcoral | 11.6 | 55.7 | 9.1 | 49.2 | 23.1 | 39.2 | 1.3 | 4.2 | 26.1 | 2.7 | 2.09 | 1.43 | 1.07 | 18.9 |
| OCN | | | | | | | | | | | | | | |
| 008 | | | | | | | | | | | | | | |
| Vcoral | 22.2 | 100.6 | 9.4 | 66.2 | 22.6 | 95.1 | ND | 9.6 | 26.8 | 8.4 | 2.01 | 2.37 | 2.42 | 10.4 |
| OCN | | | | | | | | | | | | | | |
| 014 | | | | | | | | | | | | | | |
| Vpara | 2.3 | 15.9 | 1.7 | 11.0 | 2.6 | 2.5 | 2.0 | 2.0 | 7.3 | 3.8 | 0.63 | 0.17 | 0.68 | 2.8 |
| RIMD | | | | | | | | | | | | | | |
| 2210633 | | | | | | | | | | | | | | |
| Vpara | 3.0 | 18.9 | 12.5 | 12.7 | 2.8 | 3.5 | ND | 2.4 | 8.4 | 4.5 | 0.87 | 0.27 | 1.28 | 6.4 |
| D4 | | | | | | | | | | | | | | |
| Vpara | 1.7 | 11.8 | 1.6 | 8.1 | 3.0 | 4.0 | ND | 2.1 | 6.7 | 3.3 | 1.03 | 0.32 | 1.04 | 8.1 |
| 12- | | | | | | | | | | | | | | |
| 297/B | | | | | | | | | | | | | | |
| Vvul | 0.5 | 3.4 | 0.4 | 2.7 | 0.7 | 0.9 | 0.5 | 0.4 | 1.9 | 0.7 | 0.00144 | 0.00196 | 0.00075 | 0.7 |
| ATCC | | | | | | | | | | | | | | |
| 27562 | | | | | | | | | | | | | | |
| Vvul | 2.1 | 29.6 | 2.3 | 22.0 | 6.1 | 5.9 | ND | 2.1 | 11.5 | 4.6 | 0.03392 | 0.02949 | 0.06046 | 4.9 |
| CMPC6 | | | | | | | | | | | | | | |
| Vvul | 0.9 | 6.5 | 0.8 | 4.6 | 1.3 | 1.5 | ND | 0.5 | 3.0 | 1.0 | 0.00584 | 0.00378 | 0.00546 | 3.2 |
| YJ016 | | | | | | | | | | | | | | |

^{a}Vcamp, V. campbellii; Vcoral, V. coralliilyticus; Vpara, V. parahaemolyticus; Vvul, V. vulnificus
^{b}ND, not determined.

TABLE 4
ITC analysis of LuxR/SmcR interaction with inhibitors.

| | | ΔH | | −TΔS | ΔG | ΔS |
|---|---|---|---|---|---|---|
| | K_{d} (M) | (kJ/mol) | Kₐ (M⁻¹) | (kJ/mol) | (kJ/mol) | (J/mol · K) |
| LuxR | 5.13E−07 | −136.10 | 2.30E+06 | 99.99 | −36.09 | −335.40 |
| 10B | | | | | | |
| SmcR | 2.27E−07 | −142.10 | 4.85E+06 | 104.05 | −38.04 | −348.97 |
| 10B | | | | | | |
| SmcR | 7.38E−07 | −81.91 | 1.36E+06 | 46.91 | −35.00 | −157.30 |
| Qstatin | | | | | | |

Exemplary chemical entities useful in methods of the description will now be described by reference to illustrative synthetic schemes for their general preparation below and the specific examples that follow. Artisans will recognize that, to obtain the various compounds herein, starting materials may be suitably selected so that the ultimately desired substituents will be carried through the reaction scheme with or without protection as appropriate to yield the desired product. Alternatively, it may be necessary or desirable to employ, in the place of the ultimately desired substituent, a suitable group that may be carried through the reaction scheme and replaced as appropriate with the desired substituent. Furthermore, one of skill in the art will recognize that the transformations shown in the schemes below may be performed in any order that is compatible with the functionality of the particular pendant groups.

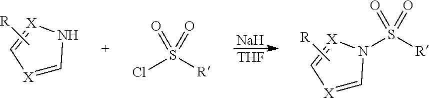

For aromatic amines: To a solution of amine (6 mmol) in tetrahydrofuran (15 mL) was added sodium hydride (60% in oil, 320 mg, 8 mmol) at room temperature, and the mixture was stirred for 10 min. A solution of sulfonyl chloride (4 mmol) in tetrahydrofuran (5 mL) was added, and the mixture was stirred for an additional 30 min. The reaction mixture was diluted with 20 mL water and extracted with ethyl acetate (3×20 mL). The extract was washed with 20 mL saturated NaCl (brine), dried over anhydrous magnesium sulfate, and concentrated under reduced pressure. The resulting crude product was purified by column chromatography (Hexanes:EtOAc).

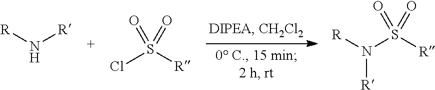

For aliphatic amines: A solution of sulfonyl chloride (4 mmol) in dichloromethane (1.0 mL) was added dropwise to a solution of amine (8 mmol) and diisopropylethylamine (1.5 mL, 8 mmol) in dichloromethane (10 mL) at 0° C. The reaction was allowed to stir at 0° C. for 15 min and then allowed to warm to room temperature. After 2 h, the resulting solution was washed with saturated sodium bicarbonate (20 mL), water (20 mL), 1 N HCl (20 mL) and brine (20 mL). The organic layer was dried with magnesium sulfate, and solvent was removed under reduced pressure. The resulting crude product was purified by column chromatography (Hexanes/EtOAc).

### Overall Sequence:

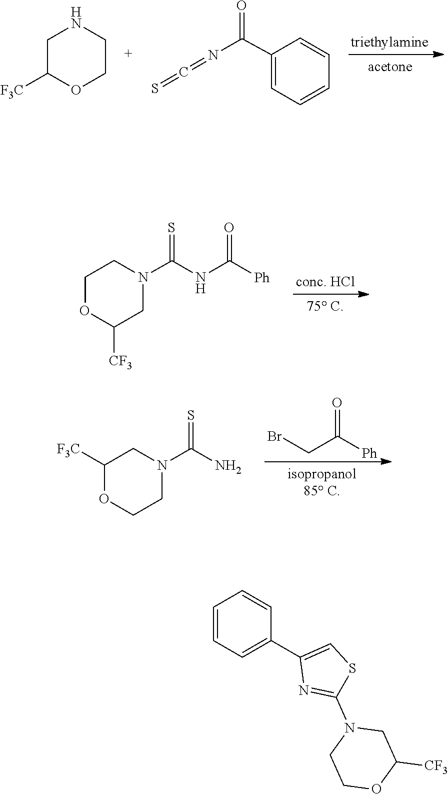 [SMILES: FC(F)(F)C1CNCCO1 ; O=C(N=C=S)c1ccccc1 ; O=C(NC(=S)N1CCOC(C(F)(F)F)C1)c1ccccc1 ; NC(=S)N1CCOC(C(F)(F)F)C1 ; O=C(CBr)c1ccccc1 ; FC(F)(F)C1CN(c2nc(-c3ccccc3)cs2)CCO1]

### Procedure for Step 1:

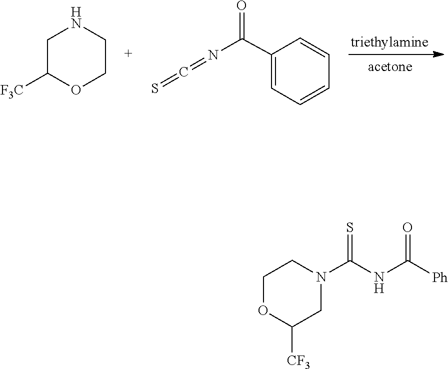 [SMILES: FC(F)(F)C1CNCCO1 ; O=C(N=C=S)c1ccccc1 ; O=C(NC(=S)N1CCOC(C(F)(F)F)C1)c1ccccc1]

For P2065 E16: A solution of 2-(trifluoromethyl)morpholine (0.50 mmol, 1 equiv) in acetone (0.50 mL) was allowed to stir under nitrogen. Triethylamine (0.75 mmol, 1.5 equiv) was added, and the resulting solution was allowed to stir at room temperature for 30 min. The solution was cooled to 0° C., and benzoyl isothiocyanate (1.0 mmol, 2.0 equiv) was added dropwise. The resulting solution was allowed to stir for 30 min at 0° C., then quenched with 1.0 mL of water. The mixture was extracted with ethyl acetate (2×5 mL). The combined organic layers were dried (Na₂SO₄) and concentrated in vacuo. The residue was purified using silica gel column chromatography with 5:1 hexanes:ethyl acetate.

### Procedure for Step 2 (Li, Ban et al. 2014):

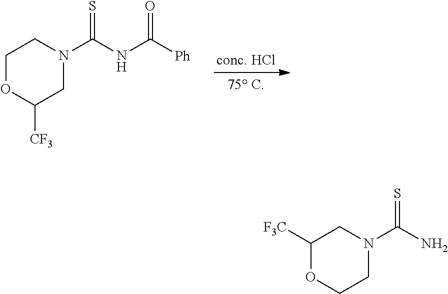 [SMILES: O=C(NC(=S)N1CCOC(C(F)(F)F)C1)c1ccccc1 ; NC(=S)N1CCOC(C(F)(F)F)C1]

Concentrated HCl (2 mL) was added to N-(2-(trifluoromethyl)morpholine-4-carbonothioyl)benzamide (0.32 mmol, 1.00 equiv) until it fully dissolved. An air condenser was added, and the resulting solution was allowed to stir for 1.5 hours at 75° C., after which time it was quenched with 10 mL ice water. A solution of 50% sodium hydroxide (10 mL) was added, and the mixture was extracted with 1:1 ethyl acetate:hexanes (3×30 mL). The combined organic layers were washed with water (20 mL), dried (Na₂SO₄), and concentrated in vacuo. The residue was purified using silica gel column chromatography with 1:1 hexanes:ethyl acetate.

### Procedure for Step 3:

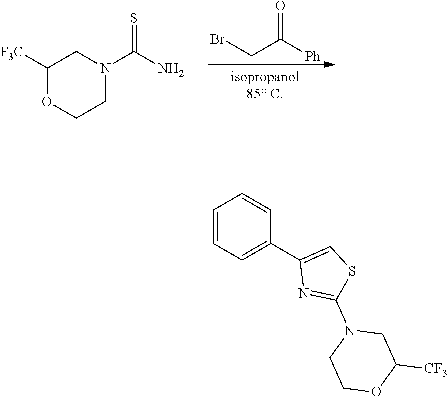 [SMILES: NC(=S)N1CCOC(C(F)(F)F)C1 ; O=C(CBr)c1ccccc1 ; FC(F)(F)C1CN(c2nc(-c3ccccc3)cs2)CCO1]

A solution of 2-bromoacetophenone (0.16 mmol, 1 equiv) of 2-(trifluoromethyl)morpholine-4-carbothioamide (0.16 mmol, 1 equiv) in isopropanol (5 ml) was heated to 85° C. and allowed to stir at that temperature for 4 hours. Upon cooling to room temperature, the organic solvent was removed under reduced pressure to produce pure desired product.

A bioassay was developed to monitor LuxR function that consists of a dual-color reporter plasmid (pJV064) that reports both activities: the luxCDABE promoter is activated by LuxR and drives expression of gfp, and the 05222 promoter is repressed by LuxR and drives expression of mCherry (van Kessel, Ulrich et al. 2013). This reporter plasmid was used in an E. coli strain that also contains a plasmid (pKM699) expressing LuxR from its native promoter. E. coli cultures containing these two plasmids were grown at 30° C. in LB medium with chloramphenicol (10 μg/ml) and tetracycline (10 μg/ml) to select for the two plasmids. Compounds were added to the cultures at a range of concentrations, and the cultures were incubated for 16 hours shaking at 30° C. In a culture that is untreated with molecule, GFP levels are high and mCherry levels are low. As a positive control, Qstatin (1-((5-bromothiophen-2-yl)sulfonyl)-1H-pyrazole), which has been shown to inhibit the LuxR protein homolog called SmcR in Vibrio vulnificus (Kim, Jang et al. 2018) was assayed.

Qstatin inhibits LuxR activity in the bioassay: GFP levels decrease and mCherry levels increase to the same amount as a control strain lacking the pKM699 plasmid. The inhibitory concentration (IC₅₀) of Qstatin in the bioassay is ˜5-10 μM. The compounds identified in FIG. 3A inhibited LuxR activation and/or repression but did not affect growth of the bacteria (Table 2). P0053118 and P2065 E16 have similar IC₅₀ values to Qstatin in the bioassay, whereas P0074 H04 is a worse inhibitor with a higher IC₅₀ value (˜35 μM).

The compounds identified in (FIG. 3) were assayed. Compounds containing an imidazole ring (class B) were not active given that the structure is highly similar to pyrroles and pyrazoles (classes A and C). Methyl substitution at the three position of the pyrazole did not alter activity compared to Qstatin, however methyl substitution at both the 3- and 5-position of the pyrazole eliminated activity (class 9). In addition, in most cases, the presence/absence of Br or Cl atoms on the thiophene ring did not alter activity. For example, Qstatin, 1B, and 1C have similar activities, and 10A, 10B, and 10C have similar activities. However, for the class 8 group, 8B does not show inhibition whereas 8A and 8C do. The conformation of the sulfonamide appears to be critical because substitution with a carbonyl eliminated activity of that class of compounds (classes F and G). The most potent molecules are compounds 10A, 10B, and 10C, all of which contain a phenyl group at the 3-position of the pyrazole and vary in the presence or absence of bromine or chlorine on the thiophene ring. The IC₅₀ values for 10A, 10B, and 10C are ˜1-2 μM. The 8A and 8C compounds have IC₅₀ values similar to Qstatin (10-20 μM), and the class 3 and class 1 compounds are worse inhibitors than Qstatin.

Qstatin has been shown to be an effective inhibitor of SmcR in vitro and in vivo (Kim, Jang et al. 2018). It also inhibits pathogenesis in V. campbellii, V. parahaemolyticus, and V. vulnificus in a shrimp infection assay, likely through inhibition of the LuxR-type protein in these strains (Kim, Jang et al. 2018). To assess the activity of our panel of sulfonamide inhibitors against other vibrios, we assayed five Vibrio species: V. campbellii BB120 (ATCC BAA-1116), V. coralliilyticus OCN008, V. cholerae E7946, V. parahaemolyticus RIMD2210633, and V. vulnificus ATCC 27562. A plasmid reporter containing the V. campbellii luxCDABE genes that produce bioluminescence has been used in many studies to measure quorum sensing regulation in vibrios because the LuxR-type protein in these species activates the luxCDABE promoter in response to quorum sensing signaling (Miller, Skorupski et al. 2002, Lenz, Miller et al. 2005, Jung, Hawver et al. 2016, Kim, Jang et al. 2018, Simpson, Podicheti et al. 2019). V. campbellii contains the luxCDABE locus; in the other four Vibrio species, we introduced the luxCDABE locus and native promoter onto a plasmid (pCS18) or the luxCDABE promoter driving expression of gfp (pCS19).

Each Vibrio strain was grown in LB or LM medium (LB with 200 mM NaCl) with kanamycin to select for the plasmids shaking at 30° C. for 16 hours in the presence of compounds at a range of concentrations. We observed that the 10A, 10B, and 10C molecules are the most inhibitory in each Vibrio strain except V. cholerae, and these have ˜10-fold lower IC₅₀ values than Qstatin. 8A has a similar IC₅₀ to Qstatin in each strain. However, there was a vast difference in the IC₅₀ for each molecule when compared across the five strains. V. vulnificus exhibited very low IC₅₀ values for all the molecules, whereas V. cholerae was not inhibited by any compound. Using 10B as an example, the IC₅₀ values were orders of magnitude different: V. vulnificus (0.04 μM), V. campbellii (0.14 μM), V. parahaemolyticus (0.28 μM), and V. coralliilyticus (2.2 μM). The large variation across strains was also true for Qstatin: V. vulnificus (0.40 μM), V. campbellii (1.3 μM), V. parahaemolyticus (2.8 μM), and V. coralliilyticus (3.6 μM). However, in every strain, 10A, 10B, and 10C were better inhibitors than Qstatin.

Portion of this study has aimed to test a panel of inhibitors against quorum sensing, a non-essential cell signaling pathway that controls pathogenesis in Vibrio species. Using a previously established dual-color bioassay, we screened thousands of compounds and synthesized a broad panel to find potent inhibitors of the quorum sensing master transcription factor LuxR. We observed that some of our key candidates contain sulfamide/sulfonamide heterocycles, but there exists a vast range of inhibition between these various compounds. Using the best candidate inhibitor PTSP, we tested its efficacy against five vibrios in vivo and found that it is most effective towards V. vulnificus, followed by V. campbellii, V. parahaemolyticus, V. coralliilyticus, and not effective against V. cholerae. The efficacy observed in inhibition of bioluminescence reporter assays was mimicked in protease assays where measurable. These results show that PTSP blocks all measured activities of LuxR proteins. This result is comparable to what was observed for the thiophenesulfonamide Qstatin, which blocks SmcR regulation of genes across the V. vulnificus genome to similar levels as a ΔsmcR strain. Thus, our results show that thiophenesulfonamides are broadly inhibitory of LuxR activities in multiple Vibrio species.

We were intrigued by the finding that the compounds we tested have no effect on HapR from V. cholerae. Our data show that HapR resistance is due to amino acid residue differences in the putative ligand binding pocket. We focused on differences in the residues in the Qstatin binding pocket across five Vibrio species, though substitution of single amino acids in HapR are not sufficient to render the protein sensitive to PTSP. Interestingly, V. cholerae also has a very different pathogenic life cycle compared to other vibrios. Pathogenesis in the human host caused by V. cholerae occurs at LCD, where the bacterial cells attach to intestinal epithelial cells through the toxin co-regulated pilus and grow as a biofilm, producing cholera toxin. Growth of the population and accumulation of autoinducers drives inhibition of biofilms through various regulatory mechanisms, cleavage from the host epithelium by the HapA protease, and the cells are shed back into the marine environment. This poses some intriguing evolutionary questions about V. cholerae pathogenesis and growth in the environment and the selective pressures that may have driven differences in amino acid conservation between HapR and other LuxR-family proteins. This could underscore the stark contrast in efficacy of inhibitors against HapR and other Vibrio LuxR-type regulators that we observed in this study.

Our modelling experiments successfully predicted the efficacy of the 10A, 10B (PTSP), and 10C molecules, which are the most potent LuxR inhibitors identified thus far. However, it is also clear that the modelling could not reliably predict every critical amino acid contact.

### Bacterial Strains and Media

E. coli strains DH10B and S17-1λpir were used for cloning, and BL21(DE3) was used for overexpression of LuxR and SmcR proteins. All E. coli strains, V. cholerae strains, and derivatives were grown in Lysogeny Broth (LB) at 30° C. shaking at 275 RPM in LB media with the appropriate antibiotic. V. campbellii, V. parahaemolyticus, V. coralliilyticus, and V. vulnificus strains and derivatives were grown shaking at 275 RPM at 30° C. in Luria Marine (LM) medium (LB with 2% NaCl) with appropriate antibiotics. Antibiotics were used at the following concentrations: kanamycin 50 μg/mL or 250 μg/mL (E. coli or Vibrio, respectively), chloramphenicol 10 μg/mL, ampicillin 100 μg/mL, gentamicin 100 μg/mL, and tetracycline 10 μg/mL.

### Molecular Methods

All PCR reactions were performed using Phusion HF polymerase (NEB). T4 polynucleotide kinase (T4 PNK) used in EMSAs and all other enzymes were purchased from NEB and used according to manufacturer's instructions. Site-directed mutagenesis for construction of plasmids expressing mutant proteins was carried out using the Agilent QuikChange II XL Site-Directed Mutagenesis Kit. All oligonucleotides were purchased from Integrated DNA Technologies (IDT).ALL plasmid constructs were confirmed by DNA sequencing (Eurofins). Cloning details for plasmids are available upon request. Western blot analysis was performed as previously described (van Kessel et al., 2013b), except that new anti-LuxR antibodies were generated through Cocalico against purified LuxR protein. In addition, the western blots were probed with anti-RpoB (Neoclone).

RNA Analysis by qRT-PCR

To collect RNA samples, cells were grown at 30° C. shaking at 275 RPM to an OD600 of approximately 0.2. Then cells were induced with 50 μM IPTG and grown under the same conditions until cells reached an OD600 of approximately 1, at which 5 mL of cells were collected by centrifugation and frozen in liquid N2. RNA was extracted using a Trizol/chloroform extraction protocol previously described (Rutherford et al., 2011). Quantitative real-time PCR (qRT-PCR) was performed as previously described (Chaparian et al., 2016). Samples were normalized to the internal standard hfq gene. The ΔΔCT values were used to analyze data from three independent biological replicates. Symbols on graphs represent the mean values and error bars represent the standard deviations. All statistical analysis was performed with functions from GraphPad Prism version 8.

### Compound Synthesis and Purchase

Some compound were synthesized by methods disclosed herein and those know in the art, Compounds P0053 O05 and P1117 F20 were purchased from Lab Network, P1120 D05 and P0074 N08 were purchased from EnamineStore, and P0053 I18 was purchased from ChemDiv.

E. coli Bioassay

The dual-promoter fluorescence reporter assays were performed using E. coli strain DH10B containing two plasmids: 1) plasmid pJV064 containing the P_{luxC} fused to GFP and P₀₅₂₂₂fused to mCherry to assess LuxR transcriptional regulation, and 2) plasmid pKM699 expressing V. campbellii luxR under control of its native promoter or empty vector pLAFR2. Overnight E. coli cultures containing either pKM699 or pLAFR2 and the pJV064 reporter were diluted 1:100 into LB with chloramphenicol and tetracycline and aliquoted into black-welled, clear-bottomed 96-well plates (150 ml final volume). Compounds were resuspended in DMSO and added to E. coli cultures at varying concentrations, or DMSO was added as a negative control at equal volumes. 96-well plates were covered in microporous sealing tape and grown for 16 hours shaking at 275 RPM at 30° C. The OD₆₀₀ and fluorescence (both GFP and mCherry) were measured on a BioTek Cytation plate reader.

In E. coli assays expressing the five different LuxR-type proteins (luxR, smcR, opaR, vcpR, hapR), slight changes to the protocol were implemented. The luxR genes were cloned under control of the IPTG-inducible Ptac promoter in plasmid pMMB67EH-kanR. E. coli DH10B strains containing one of these plasmids and the pJV064 reporter were diluted 1:1,000 into LB with chloramphenicol and kanamycin and aliquoted into black-welled, clear-bottomed 96-well plates (150 ml final volume). Strains expressing hapR or smcR required 50 μM IPTG to observe activation of the GFP reporter; IPTG was not added to strains expressing luxR, vcpR, or opaR. Strains were incubated with either 25 μM PTSP or DMSO (equal volumes) in 96-well plates covered in microporous sealing tape and grown for 16 hours shaking at 275 RPM at 30° C. The OD₆₀₀ and fluorescence (both GFP and mCherry) were measured on a BioTek Cytation plate reader.

### Protein Purification, Electrophoretic Mobility Shift Assays (EMSAs), and Isothermal Titration Calorimetry (ITC)

SmcR and LuxR were purified as described previously. EMSAs were conducted as described previously using oligonucleotides corresponding to the luxC and vvpE promoter sequences. ITC analysis was conducted using purified LuxR or His-tagged SmcR, each in gel-filtration buffer (25 mM Tris pH 7.5, 300 mM NaCl, 0.5 mM EDTA, 1 mM DTT, 2% DMSO). All molecules analyzed were diluted in the same buffer. The samples were degassed for 10 min by vacuum aspiration. Titrations were performed on a Nano-ITC (TA Instruments). SmcR in the syringe (100 μM dimer) or LuxR in the syringe (77.4 μM dimer) was titrated against 20 μM 10B (PTSP) or Qstatin in the reaction cell with 35×1 μl injections for PTSP or 23×2μl for Qstatin, stirring at 250 RPM, 120 seconds between injections, and temperature held at 25° C. Thermograms were analyzed using NanoAnalyze software with the independent binding model.

### Assaying Compounds in Vibrio Cultures

Vibrio strains were inoculated in 5 ml LM (or LB for V. cholerae) overnight at 30° C. shaking at 275 RPM with kanamycin (100 mg/ml) or gentamicin (15 mg/ml) to select for the PluxC-gfp reporter plasmids pCS19 or pCS42, respectively. Cultures were back-diluted 1:1,000 in LB or LM with antibiotics, and the cell mixture was aliquoted into black-welled, clear-bottomed 96-well plates. Compounds were titrated into the wells (4-fold dilution series; final volume of 150 ml). DMSO was added as a negative control at equal volumes into control reactions. 96-well plates were covered in microporous sealing tape and grown for 16 hours shaking at 275 RPM at 30° C. The OD₆₀₀ and GFP fluorescence or bioluminescence were measured on a BioTek Cytation plate reader.

### Protease Assays

Protease assays were conducted with a modified version of the protocol described previously (Hasegawa & Hase, 2009). Vibrio strains were inoculated in 5 ml LM (or LB for V. cholerae) overnight at 30° C. shaking at 275 RPM. Cultures were back-diluted 1:1,000 in LB or LM, and the cell mixture was aliquoted into black-welled, clear-bottomed 96-well plates. Compounds were either added into the wells to a specific final concentration or a titration series was performed (4-fold dilution series; final volume of 150 ml; 3 technical replicates per sample). DMSO was added as a negative control at equal volumes into control reactions. 96-well plates were covered in microporous sealing tape and grown for 16 hours shaking at 275 RPM at 30° C. After incubation, the OD₆₀₀ was measured on a BioTek Cytation plate reader. The cultures were pelleted in the 96-well plate by centrifuging at 3700 RPM for 5 min at room temperature. 20 ml of the supernatant was transferred to a new clear 96-well plate. 80 ml of 1% azocasein (dissolved in dH₂O) was added to the supernatants and incubated at 37° C. for 30 min. 120 ml of 10% trichloracetic acid was added to the reaction, and the plate was incubated on ice for 30 min, then centrifuged at 3700 RPM for 5 min at room temperature. 80 ml of the protease reaction was transferred to a new clear 96-well plate, and 20 ml of 1.8N NaOH was added. The OD₄₂₀ was measured on a BioTek Cytation plate reader. Protease activity was calculated by dividing OD₄₂₀ by OD₆₀₀. Each assay was performed in biological triplicates.

### Autodock Vina Modeling and Analyses

All docking experiments were performed using Autodock Vina (Trott & Olson, 2010) or Webina, the web browser-supported version (Kochnev et al., 2020). Xray crystal structures of apo SmcR (PDB ID 3KZ9) and HapR (PDB ID 2PBX) were used for all simulations (De Silva et al., 2007, Kim et al., 2010). Structures were prepared for docking using AutoDockTools-1.5.6 for addition of hydrogen atoms and assignment of partial charge (Morris et al., 2009). Ligand structures were similarly prepared to include using AutoDockTools which was additionally used to define torsional degrees of freedom. An approximately 14×14×14 Å box was defined surrounding the residues previously reported to form the Qstatin binding site (box size varied slightly with protein) (Kim et al., 2018). Pymol was used visualize the lowest energy solutions. The predicted binding energies from AutoDock Vina and/or Webina modeling were analyzed compared to the observed IC₅₀ values using K-means clustering in R using 3 clusters of sizes 3, 6, and 4.

Our structure-activity relationship data shows that multiple thoiphenesulfonamide-containing molecules with heterocycle variations are strong inhibitors of LuxR-type proteins in a wide-range of pathogenic Vibrio species.

Other variations or embodiments will be apparent to a person of ordinary skill in the art from the above description. Thus, the foregoing embodiments are not to be construed as limiting the scope of the claimed invention. All references disclosed are expressly incorporated by reference in in their entirety.

### REFERENCES

Ball, A. S., et al. (2017). “Quorum Sensing Gene Regulation by LuxR/HapR Master Regulators in Vibrios.” J. Bacteriol 199(19).
Ball, A. S. and J. C. van Kessel (2019). “The master quorum-sensing regulators LuxR/HapR directly interact with the alpha subunit of RNA polymerase to drive transcription activation in Vibrio harveyi and Vibrio cholerae.” Mol Microbiol 111(5): 1317-1334.
Jung, S. A., et al. (2016). “Parallel quorum sensing signaling pathways in Vibrio cholerae.” Curr Genet 62(2): 255-260.
Kim, B. S., et al. (2018). “QStatin, a Selective Inhibitor of Quorum Sensing in Vibrio Species.” MBio 9(1).
Lenz, D. H., et al. (2005). “CsrA and three redundant small RNAs regulate quorum sensing in Vibrio cholerae.” Mol Microbiol 58(4): 1186-1202.
Li, H., et al. (2014). “Discovery of small-molecule inhibitors selectively targeting the DNA-binding domain of the human androgen receptor.” J Med Chem 57(15): 6458-6467.
Miller, M. B., et al. (2002). “Parallel quorum sensing systems converge to regulate virulence in Vibrio cholerae.” Cell 110(3): 303-314.
Simpson, C. A., et al. (2019). “Diversity in Natural Transformation Frequencies and Regulation across Vibrio Species.” MBio 10(6).
van Kessel, J. C., et al. (2013). “Analysis of activator and repressor functions reveals the requirements for transcriptional control by LuxR, the master regulator of quorum sensing in Vibrio harveyi.” MBio 4(4).

1-45. (canceled)

46. A compound of Formula (I) 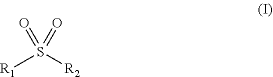
wherein R₁ and R₂ are independently selected from:
an amino, an azide, a phenylamino, an unsubstituted or an optionally substituted aryl, an unsubstituted or an optionally substituted heterocyclic ring, or an acceptable salt thereof.

47. The compound according to claim 1, wherein R₁ is not 1H-pyrazol-1-yl when R₂ is 5-bromothiphen-2-yl.

48. A compound of Formula (I) 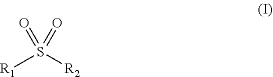
wherein R₁ and R₂ are independently selected from:
a substituted triazole or an unsubstituted triazole, a substituted pyrazole or an unsubstituted pyrazole, a substituted phenyl or an unsubstituted phenyl, a substituted thiophene, or an un-substituted thiophene,
wherein the substituted triazole, the substituted pyrazole the substituted phenyl, or the substituted thiophene are independently substituted with a methyl, an ethyl, a phenyl, a substituted phenyl, or a halogen; and
wherein R₁ is not 1H-pyrazol-1-yl when R₂ is 5-bromothiophen-2-yl.

49. The compound of claim 48, wherein the compound is selected from:
(1H-pyrazol-1-yl)(thiophen-2-yl)methanone; furan-2-yl(1H-pyrazol-1-yl)methanone; (1H-pyrrol-1-yl)(thiophen-2-yl)methanone; furan-2-yl(1H-pyrrol-1-yl)methanone; 4-(4-phenylthiazol-2-yl)-2-(trifluoromethyl)morpholine; (4-(benzo[d][1,3]dioxol-5-ylmethyl)piperazin-1-yl)(5-((4-chlorophenoxy)methyl)furan-2-yl)methanone; (1H-imidazol-1-yl)(thiophen-2-yl)methanone; 5-bromo-N-((5-bromothiophen-2-yl)sulfonyl)thiophene-2-sulfonohydrazide; N-(thiophen-2-ylsulfonyl)thiophene-2-sulfonohydrazide; and, 5-chloro-N-((5-chlorothiophen-2-yl)sulfonyl)thiophene-2-sulfonohydrazide; or, furan-2-yl(1H-imidazol-1-yl)methanone, or an acceptable salt thereof.

50. The compound of claim 48, wherein the compound is selected from: 1-(thiophen-2-ylsulfonyl)-1H-pyrazole; 1-((5-chlorothiophen-2-yl)sulfonyl)-1H-pyrazole; 1-((2-bromophenyl)sulfonyl)-1H-pyrazole; 1-(phenylsulfonyl)-1H-pyrazole; 1-((5-methylthiophen-2-yl)sulfonyl)-1H-pyrazole; 1-((2-bromophenyl)sulfonyl)-1H-pyrrole; 1-((5-bromothiophen-2-yl)sulfonyl)-1H-pyrrole; 1-(thiophen-2-ylsulfonyl)-1H-pyrrole; 1-((5-chlorothiophen-2-yl)sulfonyl)-1H-pyrrole; 1-((5-methylthiophen-2-yl)sulfonyl)-1H-pyrrole; 1-((5-bromothiophen-2-yl)sulfonyl)-1H-1,2,4-triazole; 1-((5-chlorothiophen-2-yl)sulfonyl)-1H-1,2,4-triazole; 1-((2-bromophenyl)sulfonyl)-1H-1,2,4-triazole; 1-(phenylsulfonyl)-1H-1,2,4-triazole; 1-((5-methylthiophen-2-yl)sulfonyl)-1H-1,2,4-triazole; 1-((5-bromothiophen-2-yl)sulfonyl)-3-methyl-1H-pyrazole; 3-methyl-1-(thiophen-2-ylsulfonyl)-1H-pyrazole; 1-((5-chlorothiophen-2-yl)sulfonyl)-3-methyl-1H-pyrazole; 1-((5-bromothiophen-2-yl)sulfonyl)-3-phenyl-1H-pyrazole; 3-phenyl-1-(thiophen-2-ylsulfonyl)-1H-pyrazole; 1-((5-chlorothiophen-2-yl)sulfonyl)-3-phenyl-1H-pyrazole; and 1-((5-methylthiophen-2-yl)sulfonyl)-3-phenyl-1H-pyrazole, or an acceptable salt thereof.

51. The compound of claim 48, wherein the compound is selected from: 1-((5-methylthiophen-2-yl)sulfonyl)-1H-pyrrole; 1-(phenylsulfonyl)-1H-1,2,4-triazole; 1-((5-methylthiophen-2-yl)sulfonyl)-1H-1,2,4-triazole; and 1-((5-bromothiophen-2-yl)sulfonyl)-3-methyl-1H-pyrazole or an acceptable salt thereof.

52. The compound of claim 48, wherein the compound is selected from: 1-((5-chlorothiophen-2-yl)sulfonyl)-1H-pyrazole; 1-((5-bromothiophen-2-yl)sulfonyl)-3-phenyl-1H-pyrazole; 3-phenyl-1-(thiophen-2-ylsulfonyl)-1H-pyrazole; 1-((5-chlorothiophen-2-yl)sulfonyl)-3-phenyl-1H-pyrazole; 1-((5-methylthiophen-2-yl)sulfonyl)-3-phenyl-1H-pyrazole, or an acceptable salt thereof.

53. The compound of claim 48 comprising 3-phenyl-1-(thiophen-2-ylsulfonyl)-1H-pyrazole, or an acceptable salt thereof.

54. The compound according to claim 47, wherein one or more of the compounds inhibit quorum sensing in at least one species of Vibrio.

55. The compound according to claim 54, wherein the at least one species of Vibrio is selected from the group consisting of: Vibrio vulnificus, Vibrio parahaemolyticus, and V. campbellii.

56. The compound according to claim 51, wherein the compound that inhibits quorum sensing is active at concentrations of 100 μM or less.

57. The compound according to claim 51, wherein the compound that inhibits quorum sensing is active at concentrations of 1 μM or less.

58. The compound according to claim 46, wherein the compound that inhibits quorum sensing inhibits LuxR activation and/or repression but does not affect growth of the Vibrio bacteria.

59. A method to inhibit quorum sensing in Vibrio bacteria, the method comprising the step of:
contacting at least one species of Vibrio bacteria or an environment that includes at least one species of Vibrio bacteria with a compound that inhibits quorum sensing.

60. The method according to claim 59, wherein the compound that inhibits quorum sensing inhibits LuxR activation and/or repression but does not affect growth of the Vibrio bacteria.

61. The method according to claim 59, wherein the compound that inhibits quorum sensing is a compound of Formula (I) 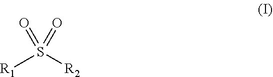
wherein R₁ and R₂ are independently selected from:
an amino, an azide, a phenylamino, an unsubstituted or an optionally substituted aryl, an unsubstituted or an optionally substituted heterocyclic ring, or an acceptable salt thereof.

62. The method according to claim 59, wherein the compound that inhibits quorum sensing is a compound of Formula (I) 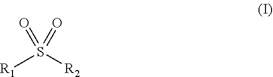
wherein R₁ and R₂ are independently selected from:
an amino, an azide, a phenylamino, an unsubstituted or an optionally substituted aryl, an unsubstituted or an optionally substituted heterocyclic ring, or an acceptable salt thereof; and
wherein R₁ is not 1H-pyrazol-1-yl when R₂ is 5-bromothiphen-2-yl.

63. The method according to claim 59, wherein the compound that inhibits quorum sensing is a compound of Formula (I) 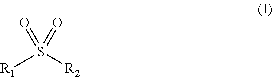
wherein R₁ and R₂ are independently selected from:
a substituted triazole or an unsubstituted triazole, a substituted pyrazole or an unsubstituted pyrazole, a substituted phenyl or an unsubstituted phenyl, a substituted thiophene, or an unsubstituted thiophene,
wherein the substituted triazole, the substituted pyrazole, the substituted phenyl or the substituted thiophene are independently substituted with a methyl, an ethyl, a phenyl, a substitute phenyl, or a halogen; and
wherein, R₁ is not 1H-pyrazol-1-yl when R₂ is 5-bromothiophen-2-yl.

64. The method according to claim 59, wherein the compound that inhibits quorum sensing is a compound selected from the group consisting of: (1H-pyrazol-1-yl)(thiophen-2-yl)methanone; furan-2-yl(1H-pyrazol-1-yl)methanone; (1H-pyrrol-1-yl)(thiophen-2-yl)methanone; furan-2-yl(1H-pyrrol-1-yl)methanone; 4-(4-phenylthiazol-2-yl)-2-(trifluoromethyl)morpholine; (4-(benzo[d][1,3]dioxol-5-ylmethyl)piperazin-1-yl)(5-((4-chlorophenoxy)methyl)furan-2-yl)methanone; (1H-imidazol-1-yl)(thiophen-2-yl)methanone; 5-bromo-N-((5-bromothiophen-2-yl)sulfonyl)thiophene-2-sulfonohydrazide; N-(thiophen-2-ylsulfonyl)thiophene-2-sulfonohydrazide; and, 5-chloro-N-((5-chlorothiophen-2-yl)sulfonyl)thiophene-2-sulfonohydrazide; and, furan-2-yl(1H-imidazol-1-yl)methanone, or an acceptable salt thereof.

65. The method according to claim 59, wherein the compound that inhibits quorum sensing is a compound selected from the group consisting of: 1-(thiophen-2-ylsulfonyl)-1H-pyrazole; 1-((5-chlorothiophen-2-yl)sulfonyl)-1H-pyrazole; 1-((2-bromophenyl)sulfonyl)-1H-pyrazole; 1-(phenylsulfonyl)-1H-pyrazole; 1-((5-methylthiophen-2-yl)sulfonyl)-1H-pyrazole; 1-((2-bromophenyl)sulfonyl)-1H-pyrrole; 1-((5-bromothiophen-2-yl)sulfonyl)-1H-pyrrole; 1-(thiophen-2-ylsulfonyl)-1H-pyrrole; 1-((5-chlorothiophen-2-yl)sulfonyl)-1H-pyrrole; 1-((5-methylthiophen-2-yl)sulfonyl)-1H-pyrrole; 1-((5-bromothiophen-2-yl)sulfonyl)-1H-1,2,4-triazole; 1-((5-chlorothiophen-2-yl)sulfonyl)-1H-1,2,4-triazole; 1-((2-bromophenyl)sulfonyl)-1H-1,2,4-triazole; 1-(phenylsulfonyl)-1H-1,2,4-triazole; 1-((5-methylthiophen-2-yl)sulfonyl)-1H-1,2,4-triazole; 1-((5-bromothiophen-2-yl)sulfonyl)-3-methyl-1H-pyrazole; 3-methyl-1-(thiophen-2-ylsulfonyl)-1H-pyrazole; 1-((5-chlorothiophen-2-yl)sulfonyl)-3-methyl-1H-pyrazole; 1-((5-bromothiophen-2-yl)sulfonyl)-3-phenyl-1H-pyrazole; 3-phenyl-1-(thiophen-2-ylsulfonyl)-1H-pyrazole; 1-((5-chlorothiophen-2-yl)sulfonyl)-3-phenyl-1H-pyrazole; and 1-((5-methylthiophen-2-yl)sulfonyl)-3-phenyl-1H-pyrazole, or an acceptable salt thereof.

66. The method according to claim 59, wherein the compound that inhibits quorum sensing is a compound selected from the group consisting of: 1-((5-methylthiophen-2-yl)sulfonyl)-1H-pyrrole; 1-(phenylsulfonyl)-1H-1,2,4-triazole; 1-((5-methylthiophen-2-yl)sulfonyl)-1H-1,2,4-triazole; and 1-((5-bromothiophen-2-yl)sulfonyl)-3-methyl-1H-pyrazole or an acceptable salt thereof.

67. The method according to claim 59, wherein the compound that inhibits quorum sensing is a compound selected from the group consisting of: 1-((5-chlorothiophen-2-yl)sulfonyl)-1H-pyrazole; 1-((5-bromothiophen-2-yl)sulfonyl)-3-phenyl-1H-pyrazole; 3-phenyl-1-(thiophen-2-ylsulfonyl)-1H-pyrazole; 1-((5-chlorothiophen-2-yl)sulfonyl)-3-phenyl-1H-pyrazole; 1-((5-methylthiophen-2-yl)sulfonyl)-3-phenyl-1H-pyrazole, or an acceptable salt thereof.

68. The method according to claim 59, wherein the compound that inhibits quorum sensing is a compound comprising 3-phenyl-1-(thiophen-2-ylsulfonyl)-1H-pyrazole, or an acceptable salt thereof.

69. The method according to claim 59, wherein the compound inhibits quorum sensing in at least one species of Vibrio.

70. The method according to claim 59, wherein at least one species of Vibrio is selected from the group consisting of: Vibrio vulnificus, Vibrio parahaemolyticus, and V. campbellii.

71. The method according to claim 59, wherein the compound that inhibits quorum sensing is active at concentrations of 100 μM or less.

72. The method according to claim 59, wherein the compound that inhibits quorum sensing is active at concentrations of 1 μM or less.

73. The method according to claim 59, further including the step of adding the compound to water used in aquaculture.

74. A method to treat or to reduce the risk for developing a Vibrio infection in a subject in need thereof, the method comprising the step of:
administering at least one therapeutically effective dose of a compound that inhibits quorum sensing in Vibrio bacteria or a pharmaceutically acceptable salt of the compound, to a patient diagnosed with or at risk for developing an infection with at least one species of Vibrio bacteria.

75. The method according to claim 74, wherein the compound that inhibits quorum sensing in Vibrio bacteria inhibits LuxR activation and/or repression but does not affect growth of the Vibrio bacteria.

76. The method according to claim 74, wherein the compound that inhibits quorum sensing is a compound of Formula (I) 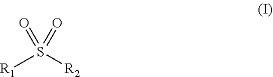
wherein R₁ and R₂ are independently selected from:
an amino, an azide, a phenylamino, an unsubstituted or an optionally substituted aryl, an unsubstituted or an optionally substituted heterocyclic ring, or an acceptable salt thereof.

77. The method according to claim 74, wherein the compound that inhibits quorum sensing is a compound of Formula (I) 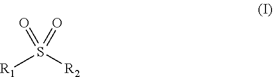
wherein R₁ and R₂ are independently selected from:
an amino, an azide, a phenylamino, an unsubstituted or an optionally substituted aryl, an unsubstituted or an optionally substituted heterocyclic ring, or an acceptable salt thereof; and
wherein R₁ is not 1H-pyrazol-1-yl when R₂ is 5-bromothiphen-2-yl.

78. The method according to claim 74, wherein the compound that inhibits quorum sensing is a compound of Formula (I) 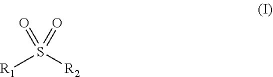
wherein R₁ and R₂ are independently selected from:
a substituted triazole or an unsubstituted triazole, a substituted pyrazole or an unsubstituted pyrazole, a substituted phenyl or an unsubstituted phenyl, a substituted thiophene or an unsubstituted thiophene,
wherein the substituted triazole, the substituted pyrazole, the substituted phenyl or the substituted thiophene are independently substituted with a methyl, an ethyl, a phenyl, a substitute phenyl, or a halogen; and
wherein, R₁ is not 1H-pyrazol-1-yl when R₂ is 5-bromothiphen-2-yl.

79. The method according to claim 74, wherein the compound that inhibits quorum sensing is a compound selected from the group consisting of: (1H-pyrazol-1-yl)(thiophen-2-yl)methanone; furan-2-yl(1H-pyrazol-1-yl)methanone; (1H-pyrrol-1-yl)(thiophen-2-yl)methanone; furan-2-yl(1H-pyrrol-1-yl)methanone; 4-(4-phenylthiazol-2-yl)-2-(trifluoromethyl)morpholine; (4-(benzo[d][1,3]dioxol-5-ylmethyl)piperazin-1-yl)(5-((4-chlorophenoxy)methyl)furan-2-yl)methanone; (1H-imidazol-1-yl)(thiophen-2-yl)methanone; 5-bromo-N-((5-bromothiophen-2-yl)sulfonyl)thiophene-2-sulfonohydrazide; N-(thiophen-2-ylsulfonyl)thiophene-2-sulfonohydrazide; and, 5-chloro-N-((5-chlorothiophen-2-yl)sulfonyl)thiophene-2-sulfonohydrazide; and, furan-2-yl(1H-imidazol-1-yl)methanone, or an acceptable salt thereof.

80. The method according to claim 74, wherein the compound that inhibits quorum sensing is a compound selected from the group consisting of: 1-(thiophen-2-ylsulfonyl)-1H-pyrazole; 1-((5-chlorothiophen-2-yl)sulfonyl)-1H-pyrazole; 1-((2-bromophenyl)sulfonyl)-1H-pyrazole; 1-(phenylsulfonyl)-1H-pyrazole; 1-((5-methylthiophen-2-yl)sulfonyl)-1H-pyrazole; 1-((2-bromophenyl)sulfonyl)-1H-pyrrole; 1-((5-bromothiophen-2-yl)sulfonyl)-1H-pyrrole; 1-(thiophen-2-ylsulfonyl)-1H-pyrrole; 1-((5-chlorothiophen-2-yl)sulfonyl)-1H-pyrrole; 1-((5-methylthiophen-2-yl)sulfonyl)-1H-pyrrole; 1-((5-bromothiophen-2-yl)sulfonyl)-1H-1,2,4-triazole; 1-((5-chlorothiophen-2-yl)sulfonyl)-1H-1,2,4-triazole; 1-((2-bromophenyl)sulfonyl)-1H-1,2,4-triazole; 1-(phenylsulfonyl)-1H-1,2,4-triazole; 1-((5-methylthiophen-2-yl)sulfonyl)-1H-1,2,4-triazole; 1-((5-bromothiophen-2-yl)sulfonyl)-3-methyl-1H-pyrazole; 3-methyl-1-(thiophen-2-ylsulfonyl)-1H-pyrazole; 1-((5-chlorothiophen-2-yl)sulfonyl)-3-methyl-1H-pyrazole; 1-((5-bromothiophen-2-yl)sulfonyl)-3-phenyl-1H-pyrazole; 3-phenyl-1-(thiophen-2-ylsulfonyl)-1H-pyrazole; 1-((5-chlorothiophen-2-yl)sulfonyl)-3-phenyl-1H-pyrazole; and 1-((5-methylthiophen-2-yl)sulfonyl)-3-phenyl-1H-pyrazole, or an acceptable salt thereof.

81. The method according to claim 74, wherein the compound that inhibits quorum sensing is a compound selected from the group consisting of: 1-((5-methylthiophen-2-yl)sulfonyl)-1H-pyrrole; 1-(phenylsulfonyl)-1H-1,2,4-triazole; 1-((5-methylthiophen-2-yl)sulfonyl)-1H-1,2,4-triazole; and 1-((5-bromothiophen-2-yl)sulfonyl)-3-methyl-1H-pyrazole or an acceptable salt thereof.

82. The method according to claim 74, wherein the compound that inhibits quorum sensing is a compounds selected from the group consisting of: 1-((5-chlorothiophen-2-yl)sulfonyl)-1H-pyrazole; 1-((5-bromothiophen-2-yl)sulfonyl)-3-phenyl-1H-pyrazole; 3-phenyl-1-(thiophen-2-ylsulfonyl)-1H-pyrazole; 1-((5-chlorothiophen-2-yl)sulfonyl)-3-phenyl-1H-pyrazole; 1-((5-methylthiophen-2-yl)sulfonyl)-3-phenyl-1H-pyrazole, or an acceptable salt thereof.

83. The method according to claim 74, wherein the compound that inhibits quorum sensing is a compound comprising 3-phenyl-1-(thiophen-2-ylsulfonyl)-1H-pyrazole, or an acceptable salt thereof.

84. The method according to claim 74, wherein the compound inhibits quorum sensing in at least one species of Vibrio.

85. The method according to claim 74, wherein at least one species of Vibrio is selected from the group consisting of: Vibrio vulnificus, Vibrio parahaemolyticus, and V. campbellii.

86. The method according to claim 74, wherein the at least one therapeutically effective dose of the compound includes from about 1 pg/kg to about 10 μg/kg.

87. The method according to claim 86, wherein the dose/kg refers to the dose per kilogram of a patient's body mass or body weight.

88. The method according to claim 74, wherein the patient is a fish.

89. The method according to claim 74, wherein the compound is added to fish feed formulations.

90. The method according to claim 89, wherein the fish feed comprises from about 1 to about 2500 mg of the compound or a physiologically acceptable derivative or salt thereof in association with and per kg of the fish feed composition.
